(19) United States
(12) Patent Application Publication
(10) Pub. No.: US 2025/0134822 A1
(43) Pub. Date: May 1, 2025

(54) NANOPARTICLE SURFACE TOPOGRAPHY MODIFIED WITH BOTTLEBRUSH BLOCK COPOLYMERS

(71) Applicant: Yale University, New Haven, CT (US)

(72) Inventors: W. Mark Saltzman, New Haven, CT (US); Julian Grundler, New Haven, CT (US); Mingjiang Zhong, Stamford, CT (US)

(21) Appl. No.: 18/692,729

(22) PCT Filed: Sep. 16, 2022
(86) PCT No.: PCT/US2022/043842

§ 371 Date: Mar. 15, 2024

Related U.S. Application Data
(60) Provisional application No. 63/245,576, filed on Sep. 17, 2021.

(51) Int. Cl. A61K 9/51; C08G 83/00

(52) U.S. Cl. CPC A61K 9/5153; C08G 83/002

(57) ABSTRACT

A method to precisely tailor the surface topography of polymeric nanoparticles having bound thereto polyalkylene oxide, based on tuning the architecture of shape-persistent amphiphilic bottlebrush block copolymer (BBCP) building blocks, has been developed. It was demonstrated that nanoparticle formation and surface topography can be controlled by systematically changing structural parameters of BBCP architecture. The surface topography of PEGylated nanoparticles (nanoparticles having PEO or PEG covalently bound thereto) significantly affects their biological performance.

### RELATED APPLICATIONS

This application claims priority to, and incorporates by reference, U.S. Provisional patent application U.S. Ser. No. 63/245,576 entitled “NANOPARTICLE SURFACE TOPOGRAPHY MODIFIED WITH BOTTLEBRUSH BLOCK COPOLYMERS” by Yale University, inventors W. Mark Saltzman, Julian Grundler, and Mingjiang Zhong, filed on Sep. 17, 2021.

### GOVERNMENT SUPPORT

This invention was made with government support under Grant DMR-2003875 awarded by the National Science Foundation and Grant Nos. U01-AI145965 and U01 AI145965 awarded by the National Institutes of Health. The government has certain rights in the invention.

### BACKGROUND OF THE INVENTION

Nanoparticle-based therapeutics and diagnostics offer immense potential in a wide range of biomedical applications. For example, drug-loaded nanoparticles have garnered great interest in drug delivery as a strategy to improve the pharmacokinetics of drugs (Banik, et al. WIREs Nanomed Nanobiotechnol 2016, 8, 271-299) However, the impact of nanoparticle-based drug delivery in clinical medicine has been limited with only a small number of FDA-approved formulations to date. (Anselmo, et al. Bioeng. Transl. Med. 2016, 1, 10-29; An Update. Bioeng. Transl. Med. 2019, 4, 1-16) Most formulations tested in clinical trials ultimately fail to show beneficial effects due to their rapid blood clearance and low drug delivery efficiency (Cho et al. Biomaterials 2012, 33, 1190-1200; Qian, et al. Polym. Chem. 2016, 7, 3300-3310.).

A widely used method to enhance the blood circulation time of nanoparticles is based on coating the surface with a dense layer of poly(ethylene glycol) (PEG), which reduces opsonization and subsequent clearance by the mononuclear phagocytosis system (Du, et al. Biomaterials 2015, 69, 1-11). While this stealthy PEG-coating effectively inhibits the adsorption of serum proteins on the nanoparticle surface via steric repulsion, the same characteristic inherently reduces their uptake into cells (Cao, et al. ACS Nano 2020, 14, 3563-3575). Several physicochemical parameters of PEG-coated nanoparticles such as size (Walkey, et al. J. Am. Chem. Soc. 2012, 134, 2139-2147), shape (Kinnear, et al. Chem. Rev. 2017, 117, 11476-11521), rigidity (Hui, et al. ACS Nano 2019, 13, 7410-7424; Mullner, et al. ACS Nano 2015, 9, 1294-1304), and surface functionality (Sutton, et al. Pharm. Res. 2007, 24, 1029-1046, Deng, et al Proc. Natl. Acad. Sci. U.S.A 2016, 113, 11453-11458) have been investigated as methods to modulate cell uptake and protein adsorption. However, only a small number of studies have focused on surface topography as a strategy to optimize nanoparticle design.

Tuning the surface topography of inorganic nanoparticles has resulted in improvements in nanoparticle pharmacokinetics, including cell uptake (Niu et al. J. Mater. Chem. B 2015, 4, 212-219; Wang, et al. Nat. Nanotechnol. 2018, 13, 1078-1086; Song, et al. J. Am. Chem. Soc. 2017, 139, 18247-18254; Chen, et al. ACS Cent. Sci. 2019, 5, 960-969; Lord, et al. Nano Today 2010, 5, 66-78; Roach, et al. J. Am. Chem. Soc. 2006, 128, 3939-3945). For example, Wang et al. ACS Cent. Sci. 2017, 3, 839-846 observed greatly enhanced circulation half-lives and cellular internalization of mesoporous silica nanoparticles with virus-mimicking spiky surface topography compared to a smooth-surfaced control.

Although surface properties are critical for nanoparticle performance, it is difficult to control the surface topography of polymer materials. Control of the surface topography of polymeric nanoparticles is a formidable challenge due to the limited conformational control of linear polymers that form the nanoparticle surface.

Despite their frequent use in biomedical applications, the effect of surface topography is poorly understood in the context of PEGylated polymeric nanoparticles. This may be in part due to the difficulty of controlling the surface topography and morphology of polymeric nanoparticles. The flexible nature of linear polymer building blocks that readily undergo conformational changes if exposed on the nanoparticle surface makes it challenging to modulate the surface without affecting their conformation. In particular, PEG chains transition from mushroom to brush conformation as their surface density increases, leading to drastically reduced serum protein adsorption and macrophage uptake (Du, et al. Biomaterials 2015, 69, 1-11). Zhou et al. ACS Nano 2018, 12, 10130-10141 demonstrated that a second PEG layer can be conjugated onto PEGylated nanoparticles to create a dynamic topographical structure that prolongs circulation times in vivo. However, their results were attributed to the combined effect of conformational fluctuations of the low-density outer PEG layer and the steric repulsion of the dense inner PEG layer rather than surface topography.

To independently evaluate the effect of surface topography on the nanoparticle's biological fate, it is critical to alter surface topography without changing the density and conformation of PEG chains on the nanoparticles surface.

It is therefore an object of the present invention to provide a method to provide controlled and stable surface topologies that prolong half-lives and/or enhance cell uptake following administration for drug delivery.

It is a further object to provide a method which is economical, reproducible, and controllable.

It is another object to provide polymeric nanoparticles having a control surface architecture formed of shape-persistent amphiphilic bottlebrush block copolymer (BBCP) building blocks, which are useful for enhanced, controlled drug delivery.

### SUMMARY OF THE INVENTION

A method to precisely tailor the surface topography of polymeric nanoparticles having bound thereto poly(ethylene oxide) polymers (“PEO” or “PEG”), based on tuning the architecture of shape-persistent amphiphilic bottlebrush block copolymer (BBCP) building blocks, has been developed. Nanoparticle formation and surface topography can be controlled by systematically changing structural parameters of BBCP architecture. BBCP terminal PEG block brush width, hydrophilic/hydrophobic block backbone length, and interfacial asymmetry are the main structural parameters of BBCP architecture to control surface topography (FIG. 1). The method uses a triblock BBCP with interfacial PEG block (i.e., triblock) to control the interfacial asymmetry/aggregation number and a terminal PEG block of different side chain length/MW to control brush width and symmetry.

In one exemplary embodiment, 1, 2.3, 3.5 kDa PEG side chains and 2.6 kDa PLA side chain were used to form the desired BBCP architecture. However, a range of up to 10 kDa can be used for side chain length. Asymmetry (ratio of interfacial PEG side chain length/MW and PLA side chain length/MW) can be any combination of side chain MW within that range. An exemplary ratio of hydrophilic/hydrophobic block backbone length (degree of polymerization, DP) is 70/10, 60/20 or 40/40 (hydrophilic/hydrophobic block backbone DP) based on a representative particle size of 100 nm, but other combinations and a longer total backbone DP (>80 DP) can be utilized.

The method controls the surface topography of PEGylated nanoparticles based on PLA-PEG BBCP building blocks to produce monodisperse nanoparticles with highly predictable surface topography by controlling a number of structural parameters. Studies were conducted with diblock BBCPs with 1) similar PEG/PLA side chain length (symmetric), 2) dissimilar PEG/PLA side chain length (asymmetric), and 3) triblock BBCPs with additional interfacial PEG side chain of different length (triblock). The surface topography of PEGylated nanoparticles (nanoparticles having PEO or PEG covalently bound thereto) significantly affects their biological performance.

Nanoparticles with a rough surface and narrow terminal brush width exhibited low protein adsorption while still maintaining high cell uptake compared to conventional smooth nanoparticles assembled from linear PLA-PEG block copolymers. The adsorption of a model protein and the uptake into model HeLa cells were closely correlated to surface roughness and BBCP terminal PEG block brush width. The use of different PEG side chain lengths for the interfacial and terminal block (i.e., triblock BBCP) to independently control aggregation number and terminal block brush width is important as is the assembly of these BBCPs into nanoparticles with controlled surface topography.

Static light scattering shows the effect of BBCP architecture on nanoparticle molar mass and aggregation number. Despite only small variations in side chain length and block backbone DP ratio, a wide range of aggregation numbers was observed. The aggregation number for the series of symmetric BBCPs with varying backbone ratio (Sy-M40, Sy-M60, Sy-M70) increased proportionally. For diblock BBCPs with constant PLA backbone length (Asy-S60, Sy-M60, Asy-L60), increasing the length of the interfacial block PEG side chain led to a drastic decrease in aggregation number from 1958 to 59 following the increase in side chain asymmetry. A similar trend was observed for the series of BBCPs with short 1.1 kDa PEG as the terminal block side chain (Asy-S60, Tri-S40-M20, Tri-S40-L20) where the aggregation number decreased as a longer interfacial block side chain was used. Triblock BBCPs had larger aggregation numbers than corresponding diblock BBCPs with a side chain length similar to the triblock interfacial block side chain.

This class of materials allows for the precise control of surface topography of polymer nanoparticles useful in drug delivery, and imaging, and diagnostics, providing for the creation of nanoparticles with better controlled interactions with proteins, cells, and tissues. The resulting products demonstrate unexpectedly better reduction of protein adsorption and enhancement of cell uptake.

### BRIEF DESCRIPTION OF THE DRAWINGS

FIGS. 1A-1C are schematics showing the synthesis via ROMP of PEG-MM and PLA-MM (FIG. 1A), the resulting symmetric, asymmetric or triblock bottlebrush architecture (FIG. 1B), followed by nanoparticle self-assembly in water and nanoparticle surface topography based on the different bottlebrush polymers (FIG. 1C).

FIG. 2A shows schematics of how polymers can be constructed by incorporation of selected blocks to product bottlebrush block copolymers which then self-assemble to form nanoparticles (FIG. 2A). Samples are categorized based on the structural parameter that is varied. The approximated simplified structure of each BBCP is depicted next to the sample name where the left part represents the PEG segments and the right part the PLA segment. (FIG. 2B). SEC chromatograms of Tri-S40-L20 after formation of first block (ROMP I), second block (ROMP II), and final BBCP

(ROMP III) are shown in FIG. 2C.

FIGS. 3A-3G are schematics of the synthesis of brush linear diblock copolymer (FIG. 3A); brush cyclic diblock copolymer (FIG. 3B); brush tadpole-A diblock copolymer (FIG. 3C); brush tadpole-B diblock copolymer (FIG. 3D); brush linear PDGE homopolymer (FIG. 3E); brush linear PTEGGE homopolymer (FIG. 3F); and brush cyclic PDGE homopolymer (FIG. 3G).

FIGS. 4A-4C are based on NMR T2 relaxation of biphasic nanoparticle shell. FIG. 4A is a schematic illustration of NMR T2 relaxation measurement procedure for biexponential fitting of methoxy (3.3 ppm) and PEG (3.6 ppm) proton signals. FIG. 4B is a schematic representation of a nanoparticle assembled from PLA-PEG BBCPs consisting of fast-relaxing inner shell and slow-relaxing outer shell. FIG. 4C is a summary of fast/slow-relaxing PEG-methoxy end groups obtained from biexponential fitting (Sy-M40; Sy-M60 and Sy-M70 backbone ratios; Asy-S60, Sy-M60, and Asy-L60 asymmetry; Asy-S60, Tri-S40-M20, and Tri-S40-L20 interfacial asymmetry; and Tri-S40M20, Sy-M60 and Tri-L40-M20 brush width). Data are presented as mean±SD (two-tailed t test, n=3 independent replicates). *P<0.05, ** P<0.01.

FIG. 5 is a schematic illustration of a nanoparticle with hydrophobic core and hydrophilic shell approximated as overlapping cylinders. The mean-line surface radius (R_{MLS}) represents the radius where the brush volume above equals the valley volume below the MLS. The inner shell sphere radius (Rss) corresponds to the radius of the overlapping portion of the bottlebrush cylinder shell.

FIG. 6A-6E are graphs showing the effect of nanoparticle surface topography on bovine serum albumin (“BSA”) adsorption and cell uptake. FIG. 6A is a graph of BSA-FITC adsorption of nanoparticles assembled from PLA-PEG BBCPs (see FIG. 4C) and a smooth nanoparticle control based on linear PLA-PEG. FIG. 6B is a graph of the impact of nanoparticle roughness on the number of BSA-FITC adsorbed per nanoparticle for 1.1 kDa, 2.3 kDa and 3.5 kDa PEG brushes. Trendlines are based on the molecular weight of the terminal block PEG side chain used. FIG. 6C is a graph of the number of BSA-FITC per nanoparticle compared to the number of accessible slow-relaxing groups obtained from NMR relaxation. FIG. 6D is a graph of the mean fluorescence intensity (MFI) of HeLa cells incubated with DiO-loaded nanoparticles as depicted in FIG. 6A. FIG. 6E is a graph comparing nanoparticle uptake into HeLa cells to the number of BSA molecules adsorbed for nanoparticles with 1.1 or 2.3 kDa PEG side chains as the terminal block. Data are presented as mean±SD (two-tailed t test, n=4 independent replicates in (a) and n=3 in (c) and (d). * P<0.05, *** P<0.001, **** P<0.0001.

FIGS. 7A and 7B are graphs showing nanoparticle tumor accumulation based on IVIS images of subcutaneous 4T1 tumors after injection with Cy5-labelled nanoparticles Asy-XL60 or Tri-S40-XL20. Asy-XL60 contains 5 kDa PEG side chains and Tri-S40-XL20 is based on 1.1 kDa PEG side chains for the terminal block and 5 kDa PEG for the interfacial middle block. FIG. 7A shows total IVIS radiant efficiency of tumor area not corrected for differences in nanoparticle brightness. FIG. 7B shows IVIS radiant efficiency normalized based on the brightness of Tri-S40-XL20. Data are presented as mean±SD with n=3 independent replicates for 1 hour, 24 hours, 48 hours, and 72 hours, and n=2 for later time points.

FIGS. 8A-8D are graphs of IVIS quantification of nanoparticle biodistribution and tumor accumulation in mice injected with Asy-XL60 (FIG. 8A) or Tri-S40-XL20 (FIG. 8B) based on organs harvested after 24 hours, 72 hours, and 144 hours. FIG. 8C is a graph of tumor radiant efficiency normalized based on the brightness of Tri-S40-XL20. FIG. 8D is a graph of total radiant efficiency of tumor relative to liver as an indicator of selective tumor accumulation. Data are presented as mean with n=2 independent replicates.

FIGS. 9A-9D are graphs of the FACS analysis of tumors harvested 24 hours, 72 hours, and 144 hours from mice after injection with Asy-XL60 or Tri-S40-XL20, measured as mean fluorescence intensity: FIG. 9A shows CD11b+F4/90+ cells corresponding to tumor-associated macrophages and FIG. 9B shows EPCAM+ cells (4T1 tumor cells). The percentage of fluorescence-positive CD11b+F4/90+ cells is shown in FIG. 9C and the percentage of fluorescence-positive EPCAM+ cells is shown in FIG. 9D. Data are presented as mean with n=2 independent replicates.

FIGS. 10A-10D are graphs of the pharmacokinetic profiles of Cy3-labelled nanoparticles formed from BBCPs (FIG. 10A, Sy-M40; FIG. 10B, Sy-M60, FIG. 10C, Sy-M70, and FIG. 10D, Asy-L60).

FIGS. 11A-11D are graphs of the circulation half-lives (over 48 hours) showing concentration in blood (μg/ml) (FIG. 11A, Tri-S40-M20; FIG. 11B, Tri-S40-L20; FIG. 11C, Tri-L40-M20, and control linear PLA-PEG, FIG. 11D.

FIGS. 12A-12C are graphs of the circulation half-life (FIG. 12A), area under the curve (FIG. 12B) and in vitro HeLa cell MFI (FIG. 12C) from 0 to 40 hours comparing in vitro HeLa cell uptake relative to circulation half-life showing concentration in blood (μg/ml) relative to control 1.1 kDa PEG brush; 2.3 kDa PEG brush and linear PLA-PEG.

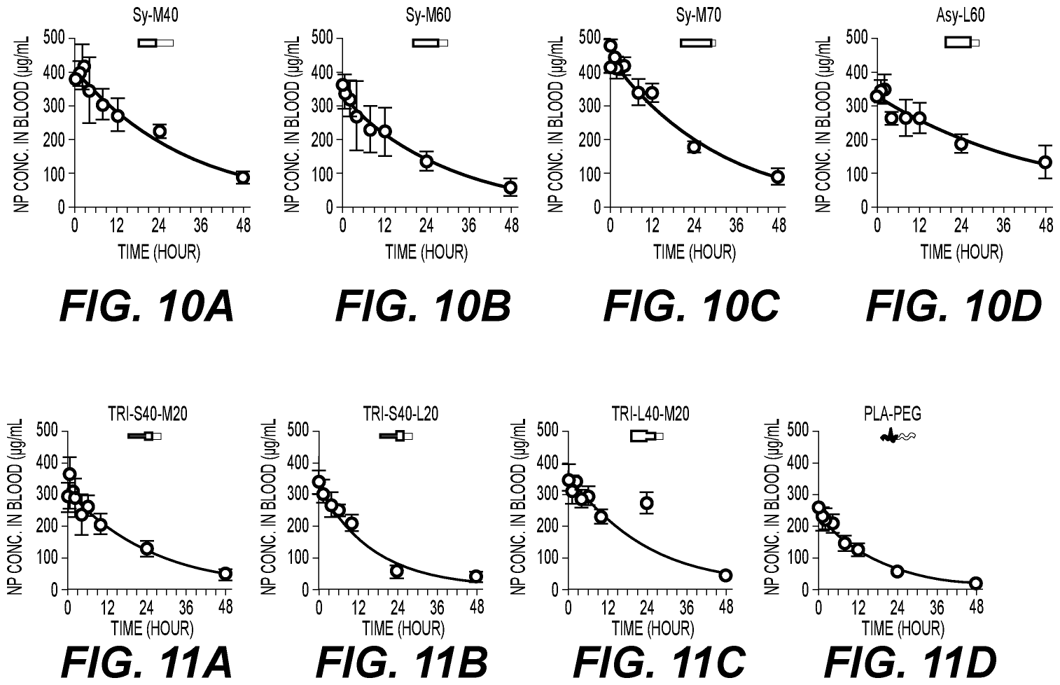

FIGS. 10-12, data are presented as mean±SD with n=4 independent replicates.

### DETAILED DESCRIPTION OF THE INVENTION

### I. Definitions

“Nanoparticle,” as used herein, generally refers to a particle of any shape having a diameter from about 1 nm up to, but not including, about 1 micron, more preferably from about 5 nm to about 500 nm, most preferably from about 5 nm to about 100 nm. Nanoparticles having a spherical shape are generally referred to as “nanospheres”.

“Mean particle size,” as used herein, generally refers to the statistical mean particle size (diameter) of the nanoparticles in a population of nanoparticles. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution,” are used interchangeably herein and describe a plurality of nanoparticles where the nanoparticles have the same or nearly the same diameter or aerodynamic diameter. As used herein, a monodisperse distribution refers to particle distributions in which 80, 81, 82, 83, 84, 85, 86, 86, 88, 89, 90, 91, 92, 93, 94, 95% or greater of the distribution lies within 5% of the mass median diameter or aerodynamic diameter.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

The term “surfactant” as used herein refers to an agent that lowers the surface tension of a liquid.

The term “therapeutic agent” refers to an agent that can be administered to prevent or treat a disease or disorder. Therapeutic agents can be a nucleic acid, a nucleic acid analog, a small molecule, a peptidomimetic, a protein, peptide, carbohydrate or sugar, lipid, or surfactant, or a combination thereof.

“Pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

“Biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Molecular weight,” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, bottlebrush polymers, also known as molecular brushes, are a special class of polymers characterized by having a “side-chain” polymer grafted to every repeating unit along the main chain polymer “backbone”. The physical size and structure of bottlebrush polymers is governed by the length (DP, or number-average molecular weight, Mₙ) of the side-chain and backbone polymers. The chemical properties (such as solubility, responsive behavior, etc) are governed primarily by side-chain polymer type or monomer selection. Together, the side-chain and backbone polymers determine bottlebrush molecular structure properties such as size, shape and stiffness. The type of side-chain polymer dictates bottlebrush functional properties, such as interaction with environment, hydrophobicity, and self-assembled structures.

Polyethylene glycol (PEG) is also known as polyethylene oxide (PEO) or polyoxyethylene (POE), depending on its molecular weight. The structure of PEG is commonly expressed as H—(O—CH₂—CH₂) n-OH. PEG, PEO, and POE refer to an oligomer or polymer of ethylene oxide. The three names are chemically synonymous, but historically PEG is preferred in the biomedical field, whereas PEO is more prevalent in the field of polymer chemistry. Because different applications require different polymer chain lengths, PEG has tended to refer to oligomers and polymers with a molecular mass below 20,000 g/mol, PEO to polymers with a molecular mass above 20,000 g/mol, and POE to a polymer of any molecular mass. PEGs are prepared by polymerization of ethylene oxide and are commercially available over a wide range of molecular weights from 300 g/mol to 10,000,000 g/mol. PEG and PEO are liquids or low-melting solids, depending on their molecular weights. While PEG and PEO with different molecular weights find use in different applications, and have different physical properties (e.g., viscosity) due to chain length effects, their chemical properties are nearly identical. Different forms of PEG are also available, depending on the initiator used for the polymerization process. The most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete.

Branched PEGs have three to ten PEG chains emanating from a central core group. Star PEGs have 10 to 100 PEG chains emanating from a central core group. Comb PEGs have multiple PEG chains normally grafted onto a polymer backbone.

The numbers that are often included in the names of PEGs indicate their average molecular weights (e.g., a PEG with n=9 would have an average molecular weight of approximately 400 Daltons, and would be labeled PEG 400). Most PEGs include molecules with a distribution of molecular weights (i.e. they are polydisperse). The size distribution can be characterized statistically by its weight-average molecular weight (M_{w}) and its number-average molecular weight (Mₙ), the ratio of which is called the polydispersity index (Ð). M_{w} and Mₙ can be measured by mass spectrometry, GPC and/or NMR.

As used herein, an asymmetric bottle brush polymer includes dissimilar PEG/PLA side chain length (asymmetric). Symmetric and asymmetric refers to length/molecular weight of the hydrophilic block polymer side chain relative to that of the hydrophobic block side chain, typically PEG/PLA side chains. If the side chains have approximately the same molecular weight, the BBCP is considered symmetric. If they are different then they are asymmetric.

Terminal PEG block brush width is defined as the length/MW of the PEG side chain used for the terminal block forming the outer nanoparticle shell/surface or more specifically the radial diameter of the terminal PEG block bottlebrush polymer. Side chain length (and width) in this study was between 1 kDa to 3.5 kDA. However, side chain lengths somewhere between 0 to 10 kDa can be used.

As used herein, an amphiphilic polymer is one which has one end formed of a hydrophilic polymer and one end formed of a hydrophobic polymer. As a result, when dispersed into a mixture of water and low water-solubility solvent such as many of the organic solvents, the hydrophilic end orients into the water and the hydrophobic end orients into the low water-solubility end.

Self-assembling refers to the use of amphiphilic polymers, alone or in mixture with hydrophilic and/or hydrophobic polymers, which orient in a mixture of aqueous and non-aqueous solvents to form nanoparticles, wherein the hydrophilic ends orient with the other hydrophilic ends and the hydrophobic ends orient with the other hydrophobic ends.

### II. Methods of Making Controlled Surface Compositions

Control of the surface topography of polymeric nanoparticles is a formidable challenge due to the limited conformational control of linear polymers that form the nanoparticle surface. A straightforward method to precisely tailor the surface topography of PEGylated polymeric nanoparticles based on tuning the architecture of shape-persistent amphiphilic bottlebrush block copolymer (BBCP) building blocks has been developed. Studies demonstrate that nanoparticle formation and surface topography can be controlled by systematically changing structural parameters of BBCP architecture. The surface topography of PEGylated nanoparticles significantly affects their performance. In particular, the adsorption of a model protein and the uptake into HeLa cells were closely correlated to surface roughness and BBCP terminal PEG block brush width.

### A. PEGylated Brush Polymers

The surface topography of PEGylated polymeric nanoparticles is based on amphiphilic bottlebrush block copolymers (BBCPs) that are composed of linear hydrophobic polymers such as the hydroxyester poly(lactic acid) (PLA) and poly(alkylene oxide) polymeric side chains, preferably PEG side chains, respectively, attached to two blocks of a polymeric backbone. Bottlebrush polymers exhibit a shape-persistent worm-like morphology due to their semi-rigid backbone, with side chains in an extended brush-like conformation given their high grafting density (Fenyves, et al. J. Am. Chem. Soc. 2014, 136, 7762-7770). In an aqueous environment, amphiphilic BBCPs readily self-assemble into micellar nanoparticles in a well-controlled manner that can be predicted from a cone-filling perspective based on the length of the hydrophobic block bottlebrush backbone and cross-sectional area of the hydrophilic-hydrophobic interface (Fenyves 2014; Unsal, et al. Macromolecules 2017, 50, 1342-1352). Manipulation of the BBCP architecture allows one to tailor the nanoparticle surface topography without changing the PEG conformation, due to the shape-persistent nature of the BBCP backbone and the fixed PEG side chains density.

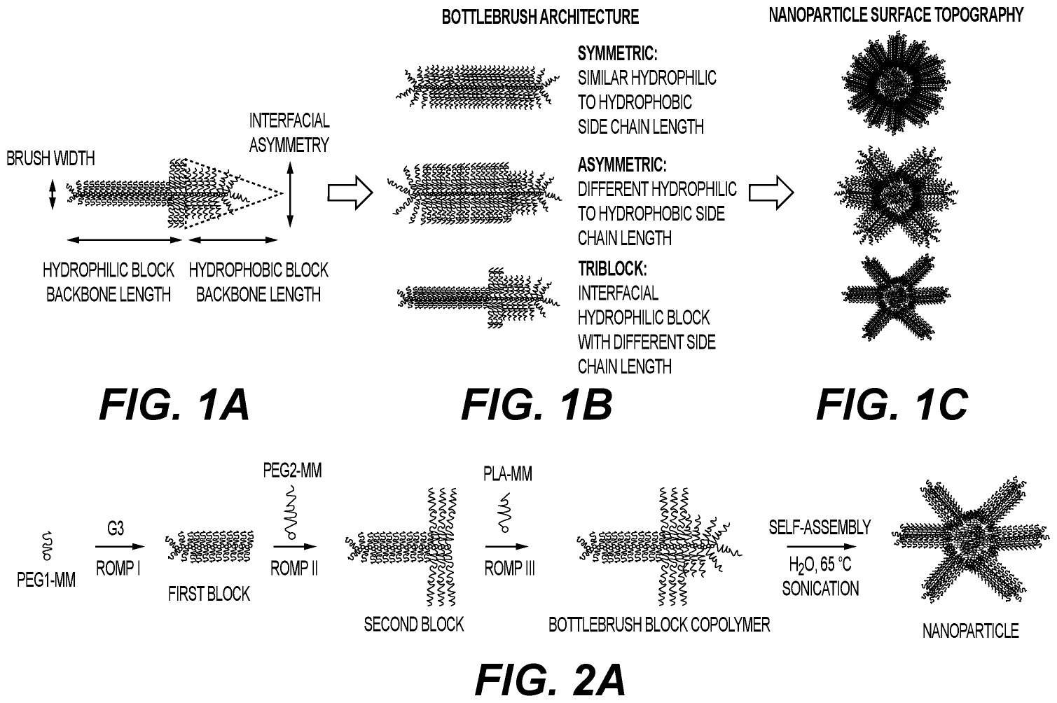

BBCP terminal PEG block brush width, hydrophilic/hydrophobic block backbone length, and interfacial asymmetry are the main structural parameters of BBCP architecture to control surface topography (FIG. 1). The architectural framework was used in a systematic study of diblock BBCPs with 1) similar PEG/PLA side chain length (symmetric), 2) dissimilar PEG/PLA side chain length (asymmetric), and 3) triblock BBCPs with additional interfacial PEG side chain of different length (triblock).

Nanoparticle cell uptake and the adsorption of a model protein is closely correlated to surface topography.

### B. Hydrophobic Polymers

Nanoparticles are preferably formed of polyoxyalkylene polymers such as PEG covalently bound to a hydrophobic polymer, preferably biodegradable and approved by the US Food and Drug Administration for administration to a human or animal, such as the polyhydroxyesters, polyanhydrides, polyhydroxyalkanoates, polycaprolactone (PCL), and polyorthoesters. Preferred polyhydroxyesters include polylactic acid (“PLA”), polyglycolic acid (“PGA”), and copolymers thereof such as PLGA.

Any number of biocompatible polymers can be used to prepare the nanoparticles. In one embodiment, the biocompatible polymer(s) is biodegradable. In another embodiment, the nanoparticles are non-degradable. In other embodiments, the nanoparticles are a mixture of degradable and non-degradable nanoparticles.

Exemplary polymers include, but are not limited to, polymers prepared from lactones, such as poly(caprolactone) (PCL); polyhydroxy acids and copolymers thereof such as poly(lactic acid) (PLA), poly(_{L}-lactic acid) (PLLA), poly(glycolic acid) (PGA), poly(lactic acid-co-glycolic acid) (PLGA), poly(L-lactic acid-co-glycolic acid) (PLLGA), poly(_{D,L}-lactide) (PDLA), poly(_{D,L}-lactide-co-caprolactone), poly(_{D,L}-lactide-co-caprolactone-co-glycolide), poly(_{D,L}-lactide-co-PEO-co-_{D,L}-lactide), poly(_{D,L}-lactide-co-PPO-co-_{D,L}-lactide), and blends thereof, polyglycerol, poly(alkyl cyanoacralate), polyurethanes, hydroxypropyl methacrylate (HPMA); polyanhydrides; polyesters; polyorthoesters; poly(ester amides); polyamides; poly(ester ethers); polycarbonates; polyalkylenes such as polyethylene and polypropylene; poly(alkylene glycol)s such as poly(ethylene glycol) (PEG) or poly(ethylene oxide)s (PEO), and block copolymers thereof such as poly(oxyalkylene oxide) (“PLURONICS®”); poly(alkylene terephthalate)s such as poly(ethylene terephthalate); ethylene vinyl acetate polymer (EVA); poly(vinyl alcohol)s (PVA); poly(vinyl ether)s; poly(vinyl ester)s such as poly(vinyl acetate); poly(vinyl halide)s such as poly(vinyl chloride) (PVC), polyvinylpyrrolidone; and polymers of acrylic acids, such as poly(methyl methacrylate) (PMMA), (jointly referred to herein as “poly(acrylic acid)s”); polydioxanone and its copolymers; polyhydroxyalkanoates; poly(propylene fumarate); polyoxymethylene; poloxamers; poly(butyric acid); trimethylene carbonate; and polyphosphazenes. Examples of preferred natural polymers include proteins such as albumin, collagen, gelatin and prolamines, for example, zein, and polysaccharides such as alginate. Copolymers of the above, such as random, block, or graft copolymers, or blends of the polymers listed above can also be used.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

In particular embodiments, the nanoparticles are formed from block copolymers containing PEG. In more particular embodiments, the nanoparticles are prepared from block copolymers containing PEG, wherein PEG is covalently bound to the terminal of the base polymer. Representative PEG molecular weights include 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, 500 kDa, and 1 MDa and all values within the range of 300 Daltons to 1 MDa. In preferred embodiments, the PEG has a molecular weight of about 5 kD. PEG of any given molecular weight may vary in other characteristics such as length, density, and branching.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

The nanoparticles are typically between 50 nm to under 500 nm, more preferably less than 250 or 300 nm, and most preferably under 100 nm.

C. Therapeutic, Prophylactic, Nutraceutical and/or Diagnostic Agent

In some embodiments, the nanoparticles have encapsulated therein, dispersed therein, and/or covalently or non-covalently associate with the surface one or more therapeutic, prophylactic and/or diagnostic agents. The therapeutic agent can be a small molecule (molecular weight under 2000 Da, more preferably under 1500 Da, most preferably under 1000 Da), protein or peptide, polysaccharide or saccharide, nucleic acid molecule and/or lipid.

Exemplary classes of therapeutic and/or prophylactic agents include, but are not limited to, analgesics, anti-inflammatory drugs, hormones, cardiovascular drugs, anti-proliferatives, such as anti-cancer agent, anti-infectious agents, such as antibacterial agents and antifungal agents, gastro-intestinal drugs, nutritional agents, vitamins and nutraceuticals. These may be vitamins, supplements such as calcium or biotin, or natural ingredients such as plant extracts or phytohormones.

In some embodiments, the agent is one or more nucleic acids. The nucleic acid can alter, correct, or replace an endogenous nucleic acid sequence. Nucleic acid therapeutics can be used to treat cancers, correct defects in genes in other pulmonary diseases and metabolic diseases affecting lung function, mitigate symptoms of neurodegeneration.

### III. Methods of Making Nanoparticles

The preferred method demonstrated in the examples is a type of self-assembly of polymers to form nanoparticles referred to as direct dissolution followed by ultrasonication. However, solvent evaporation/nanoprecipitation followed by ultrasonication can also be used and may be preferred for the encapsulation of drugs. Ultrasonication is very important to obtaining monodisperse nanoparticles using BBCPs.

Methods for making nanoparticles are known. See, for example, Teo B. M. (2016) Ultrasonic Synthesis of Polymer Nanoparticles. In: Handbook of Ultrasonics and Sonochemistry. Springer, Singapore. https://doi.org/10.1007/978-981-287-278-4_14. Nanoprecipitation is a simple method used for encapsulation of both hydrophilic and hydrophobic drugs in nanoparticles. The method results in instantaneous formation of nanoparticles, is an easy to perform technique, can be easily scaled up and is a one-step procedure. The method requires addition of two solvents that are miscible with each other and results in spontaneous formation of nanoparticles on phase separation. The first solvent is the one in which the polymer and the drug dissolves but not in the second non-solvent. A modified nanoprecipitation method utilizes a co-solvent to either increase the entrapment efficiency of the drug in nanoparticles or to reduce the mean particle size of the nanoparticles.

See also Zielińska, et al. Molecules 2020, 25, 3731, reviewing methods for production of polymeric nanoparticles. In general, two main strategies are employed, namely, the dispersion of preformed polymers or the polymerization of monomers. In order to load compounds in polymeric NPs, techniques based on the polymerization of monomers allow insertion with greater efficiency and in a single reaction step. Regardless of the method of preparation employed, the products are usually obtained as aqueous colloidal suspensions

Solvent evaporation was the first method developed to prepare polymeric NPs from a preformed polymer. In this method, the preparation of an oil-in-water (o/w) emulsion is initially required, leading to nanosphere production. First an organic phase is prepared, consisting of a polar organic solvent in which the polymer is dissolved, and the active ingredient (e.g., drug) is included by dissolution or dispersion. Then an aqueous phase which contains a surfactant such as polyvinyl acetate, PVA, is prepared. The organic solution is emulsified in the aqueous phase with a surfactant, and then it is typically processed by using high-speed homogenization or ultrasonication, yielding a dispersion of nanodroplets. A suspension of NPs is formed by evaporation of the polymer solvent, which is allowed to diffuse through the continuous phase of the emulsion. The solvent is evaporated either by continuous magnetic stirring at room temperature (in case of more polar solvents) or in a slow process of reduced pressure (as happens when using dichloromethane and chloroform). After the solvent has evaporated, the solidified nanoparticles can be washed and collected by centrifugation, followed by freeze-drying for long-term storage. This method allows the creation of nanospheres.

Emulsification/Solvent Diffusion consists of the formation of an o/w emulsion between a partially water-miscible solvent containing polymer and drug, and an aqueous solution with a surfactant. The internal phase of this emulsion consists of a partially hydro-miscible organic solvent, such as benzyl alcohol or ethyl acetate, which is previously saturated with water in order to ensure an initial thermodynamic balance of both phases at room temperature. The subsequent dilution with a large amount of water induces solvent diffusion from the dispersed droplets into the external phase, resulting in the formation of colloidal nanoparticles. Generally, this is the method used to produce nanospheres, but nanocapsules can also be obtained if a small amount of oil (such as triglycerides: C6, C8, C10, C12) is added to the organic phase. Depending on the boiling point of the organic solvent, this latter stage can be eliminated by evaporation or by filtration. This method can yield NPs with dimensions ranging from 80 to 900 nm.

Emulsification/Reverse Salting-Out is a modification of the emulsification/reverse salting-out method. The salting-out method is based on the separation of a hydro-miscible solvent from an aqueous solution, through a salting-out effect that may result in the formations of nanospheres. The main difference is the composition of the o/w emulsion, which is formulated from a water-miscible polymer solvent, such as acetone or ethanol, and the aqueous phase contains a gel, the salting-out agent and a colloidal stabilizer. Examples of suitable salting-out agents include electrolytes, such as magnesium chloride (MgCl₂), calcium chloride (CaCl₂)) or magnesium acetate [Mg(CH₃COO)₂], as well as non-electrolytes e.g., sucrose. The miscibility of acetone and water is reduced by saturating the aqueous phase, which allows the formation of an o/w emulsion from the other miscible phases. The o/w emulsion is prepared, under intense stirring, at room temperature. Then the emulsion is diluted using an appropriate volume of deionized water or of an aqueous solution in order to allow the diffusion of the organic solvent to the external phase, the precipitation of the polymer, and consequently, the formation of nanospheres. The remaining solvent and salting-out agent are eliminated by cross-flow filtration. The dimensions of the nanospheres obtained by this method vary between 170 and 900 nm. The average size can be adjusted to values between 200 and 500 nm, by varying polymer concentration of the internal phase/volume of the external phase. As the solvent diffuses out from the nanodroplets, the polymer precipitates in the form of nanocapsules or nanospheres.

Nanoprecipitation is a method frequently used for the preparation of polymeric NPs, but it also allows the acquisition of nanospheres or nanocapsules. Nanospheres are obtained when the active principle is dissolved or dispersed in the polymeric solution. Nanocapsules are obtained when the drug is previously dissolved in an oil, which is then emulsified in the organic polymeric solution before the internal phase is dispersed in the external phase of the emulsion.

In solvent evaporation, the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid nanoparticles. The resulting nanoparticless are washed with water and dried overnight in a lyophilizer. Nanoparticles with different sizes (0.5-1000 nms) and morphologies can be obtained by this method. This method is useful for relatively stable polymers like polyesters and polystyrene.

### IV. Methods and Formulations for Delivery of Therapeutic, Prophylactic or Diagnostic Agents

The nanoparticles are useful in drug delivery (as used herein “drug” includes therapeutic, diagnostic and prophylactic agents), whether injected intravenously, subcutaneously, or intramuscularly, encapsulated and administered orally, formulated as a suspension, gel, spray, other other formulation for administration to a mucosal surface (nasal, pulmonary, vaginal, rectal, buccal, sublingual), or formulated for topical delivery. The dosage is determined using standard techniques based on the drug to be delivered and the method and form of administration. The nanoparticles may be administered as a dry powder, as an aqueous suspension (in water, saline, buffered saline, etc), in a hydrogel, organogel, or liposome, in capsules, tablets, troches, or other standard pharmaceutical excipient.

These formulations are typically administered by injection, preferably intravenously.

The present invention will be further understood by reference to the following non-limiting examples.

### Example 1: Formation and Analysis of Asymmetric Bottlebrush Copolymer Structures on Nanoparticles

FIGS. 1A-1C are schematics showing the synthesis via ROMP of PEG-MM and PLA-MM (FIG. 1A), the resulting bottlebrush architecture (FIG. 1B), followed by nanoparticle self-assembly in water and nanoparticle surface topography (FIG. 1C).

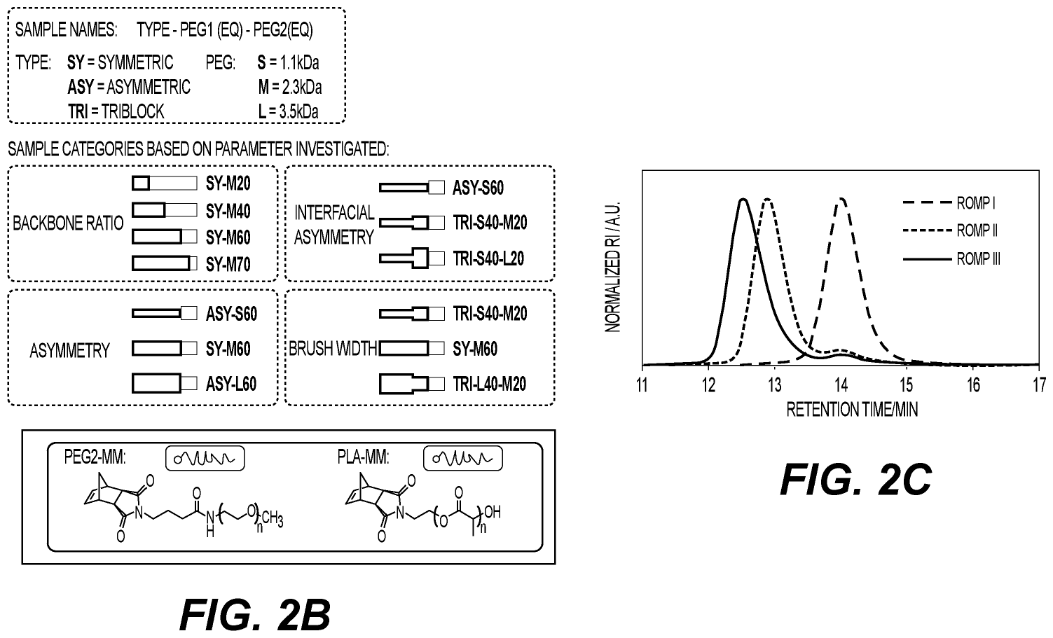

FIG. 2A shows schematics of how polymers can be constructed by incorporation of selected blocks to product bottlebrush block copolymers which then self-assemble to form nanoparticles (FIG. 2A). Samples are categorized based on the structural parameter that is varied. The approximated simplified structure of each BBCP is depicted next to the sample name where the left part represents the PEG segments and the right part the PLA segment. (2B) SEC chromatograms of Tri-S40-L20 after formation of first block (ROMP I), second block (ROMP II), and final BBCP (ROMP III) are shown in FIG. 2C.

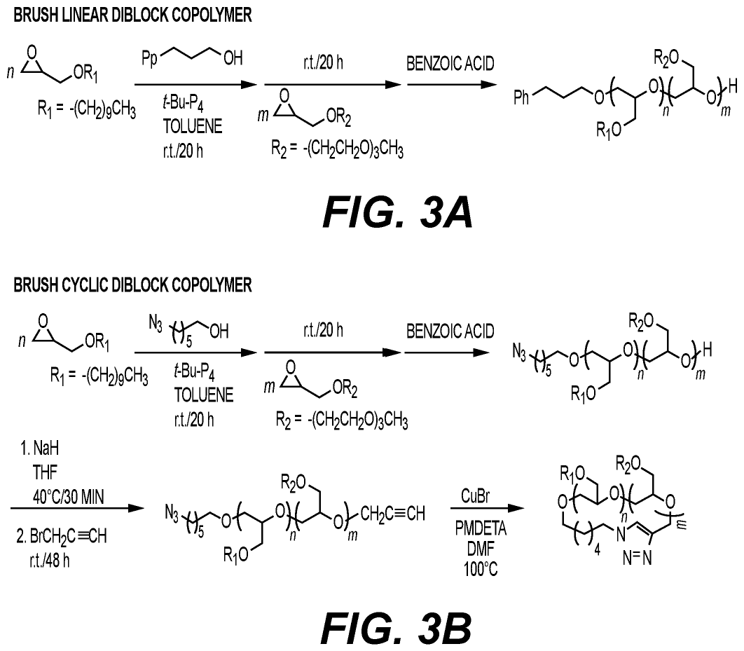

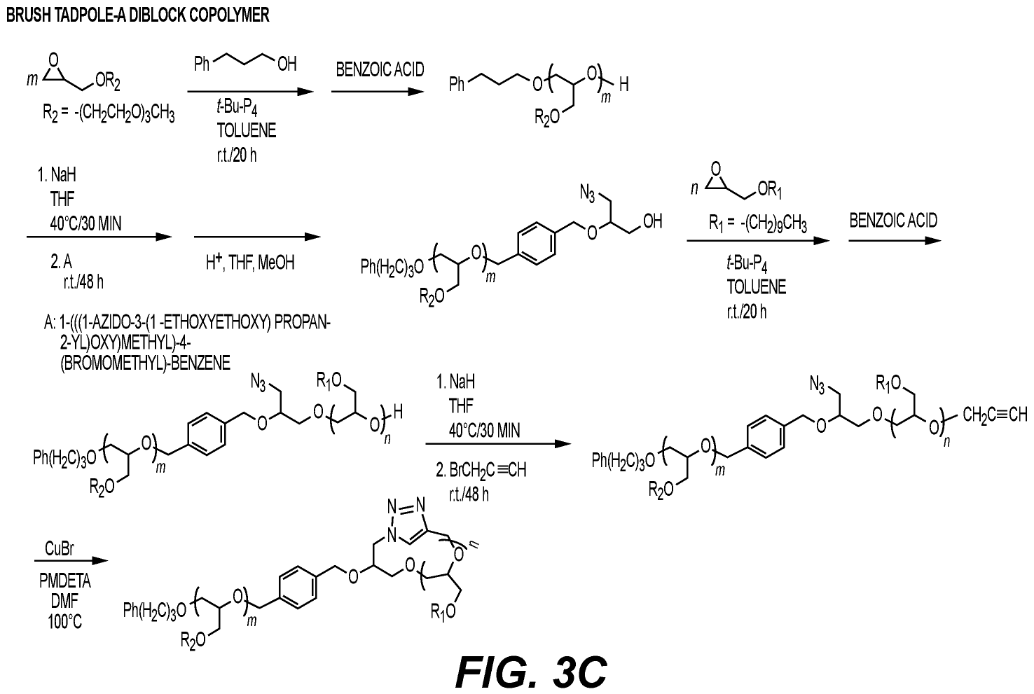

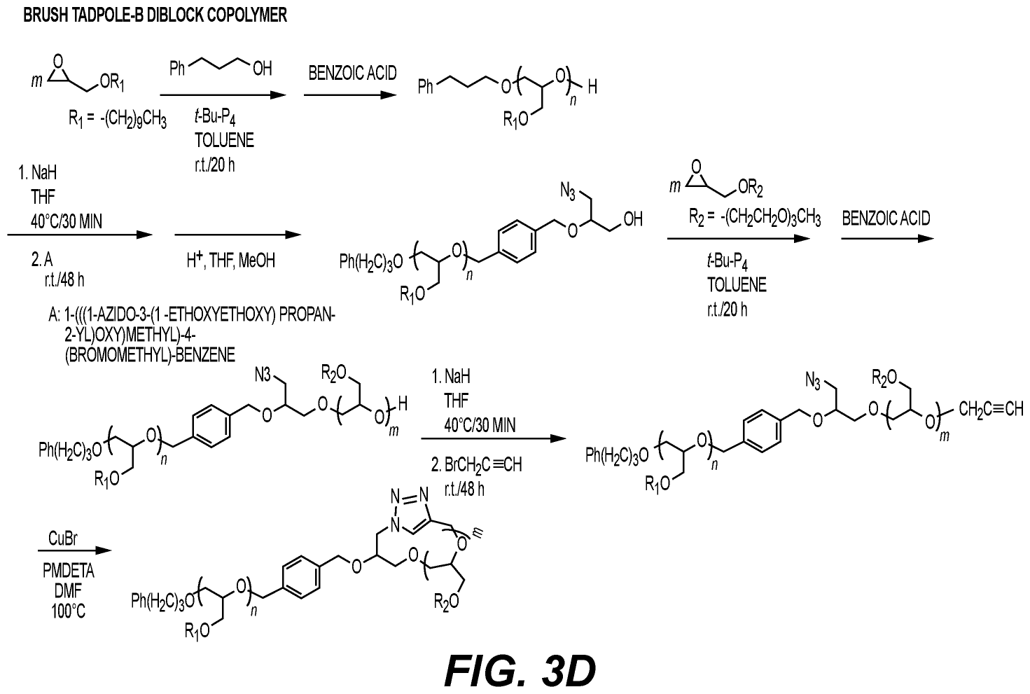

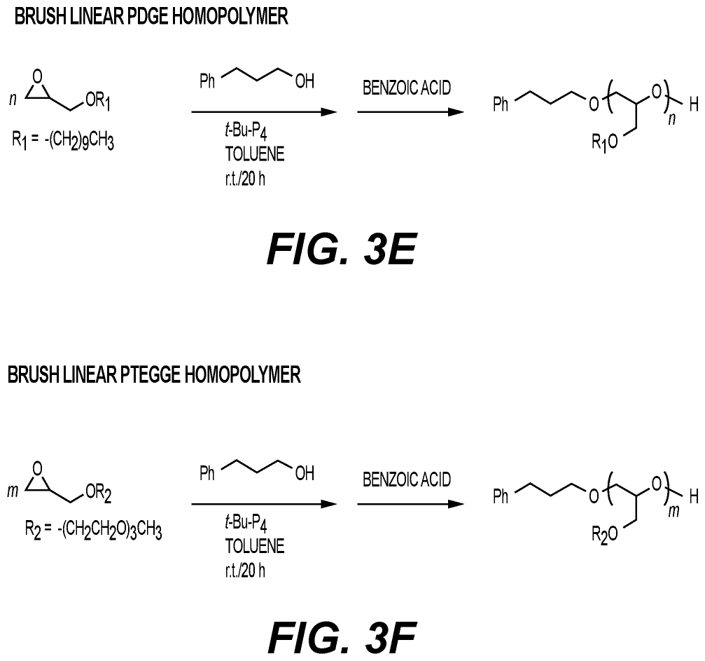

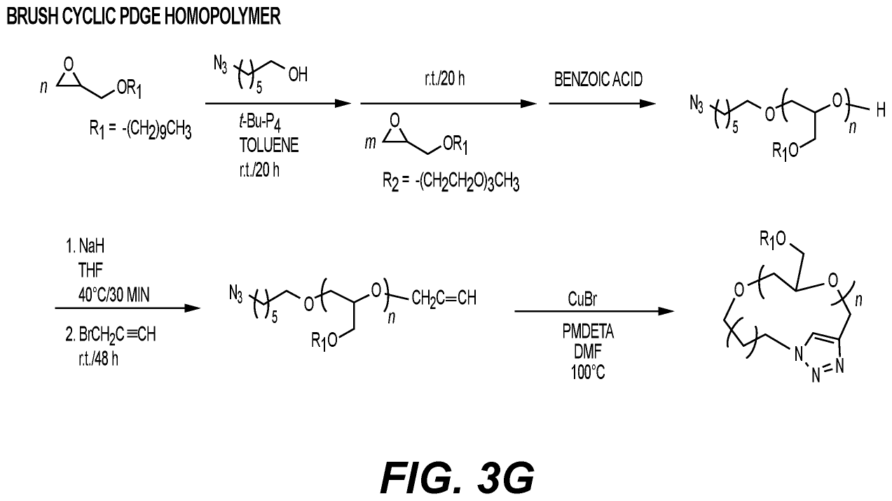

FIGS. 3A-3G are schematics of the synthesis of brush linear diblock copolymer (FIG. 3A); brush cyclic diblock copolymer (FIG. 3B); brush tadpole-A diblock copolymer (FIG. 3C); brush tadpole-B diblock copolymer (FIG. 3D); brush linear PDGE homopolymer (FIG. 3E); brush linear PTEGGE homopolymer (FIG. 3F); and brush cyclic PDGE homopolymer (FIG. 3G).

### Materials and Methods

### Materials.

Methoxy-PEG-amine (750 Da and 2 kDa), methoxy PEG (5 kDa), albumin-fluorescein isothiocyanate (BSA-FITC), and Grubbs second-generation catalyst were purchased from Sigma-Aldrich while methoxy-PEG-amine, HCl salt (3 kDa) was obtained from JenKem Technology USA. DiO dye (3,3′-dioctadecyloxacarbocyanine perchlorate) was purchased from Biotium and poly(lactic acid) (6.7 kDa and 14.3 kDa) from LACTEL. PEG-MM27 and N-(hydroxyethyl)-cis-5-norbornene-exo-2,3-dicarboximide were prepared according to Matson, et al. J. Am. Chem. Soc. 2008, 130, 6731-6733. All other solvent and reagents were purchased from commercial suppliers and used without additional purification unless otherwise noted.

### Instrumentation.

¹H NMR spectra were recorded on either a 400 MHz or 500 MHz Agilent DD2 NMR spectrometer. SEC was performed on a ThermoFisher Ultimate 3000 UHPLC equipped with two Agilent InfinityLab PolyPore columns (7.5×300 mm) connected in series at 60° C. Dimethyl formamide (DMF) with 0.1 g/mL LiBr added was used as the eluent at a flow rate of 1 mL/min. The polymer molecular weight was obtained using a T-rEX refractive index detector (Wyatt) and a Dawn Heleos II (Wyatt Technology) eight angle light scattering detector. DLS including zeta potential measurements were performed using Zetasizer Pro (Malvern Panalytical).

### PLA-MM Synthesis.

Inside a nitrogen-filled glovebox, a round-bottom flask was charged with N-(hydroxyethyl)-cis-5-norbornene-exo-2,3-dicarboximide (0.106 g, 0.51 mmol) and _{D,L}-lactide (1.010 g, 7.00 mmol). After complete dissolution of reagents in dry dichloromethane (DCM) (10 mL), 1,8-Diazabicyclo [5.4.0]undec-7-ene (DBU) catalyst (5.9 μL, 0.07 mmol) was added and the mixture stirred for 1 hour. The reaction was quenched by adding benzoic acid (54.9 mg, 0.45 mmol). The crude was purified via precipitation in hexanes (2×) and 30:70 water: methanol (2×) to obtain 829 mg white powdery polymer (74% yield).

### BBCP Synthesis.

G3 was prepared from second-generation Grubbs catalyst by stirring in toluene following Love, et al. Angew. Chemie Int. Ed. 2002, 41, 4035-4037. PEG-MM (1.1 kDa, 2.3 kDa, and 3.5 kDa) and PLA-MM stock solutions (0.05 M) were prepared by addition of dry DCM inside a nitrogen-filled glovebox. BBCPs were synthesized via ROMP. (Ring-opening metathesis polymerization (ROMP) is a type of olefin metathesis chain-growth polymerization. The driving force of the reaction is relief of ring strain in cyclic olefins (e.g., norbornene or cyclopentene).) based on the following representative procedure for Tri-S40-L20: PEG-MM stock solution (1.1 kDa, 364 μL, 18.2 μmol, 20 equiv.) was transferred to a 1.4 mL vial charged with a stir bar and freshly-prepared dark-green G3 (0.33 mg, 0.91 μmol, 1 equiv., 0.01 g/mL) in DCM added under constant stirring. After 45 min, a small aliquot (40 μL) was removed for SEC and NMR analysis followed by the dropwise addition of the second PEG-MM (3.5 kDa, 164 μL, 8.2 μmol, 10 equiv.) and additional stirring for 1 h. Another aliquot (30 μL) was taken before the dropwise addition of PLA-MM (153 μL, 7.6 μmol, 20 equiv.). The reaction mixture was allowed to stir for 90 min and then quenched with 100 μL ethyl vinyl ether.

### BBCP Self-Assembly.

BBCPs were dissolved in acetone and transferred to a round-bottom flask. For dye-loaded samples, DiO dye (0.2% w/w) in acetone was added to the BBCP solution. The solvent was slowly removed via rotary evaporation to form a polymer film at the bottom of the flask. The polymer film was dried in a vacuum oven before ultrapure water was added to obtain a polymer concentration of 1 mg/mL. The samples were stirred overnight at 65° C. followed by 15 min sonication in a sonication bath (Branson 2510) heated to 65° C. Resulting nanoparticles were filtered through a 0.45 μm polyethersulfone syringe filter and washed with ultrapure water three times through a Amicon centrifuge filter tube (100 kDa MWCO).

### Linear PLA-PEG Assembly.

PLA-PEG block copolymers were synthesized via ring-opening polymerization using methoxy-PEG (5 kDa) as macroinitiator and DBU catalyst. Synthesized PLA₈ₖ-PEG₅ₖ, PLA₁₆ₖ-PEG₅ₖ, and purchased PLA_{14.3k}, PLA_{6.7k} were dissolved in acetonitrile with a weight ratio of 40, 15, 25 and 20, respectively, to result in a total polymer concentration of 50 mg/ml. The polymer solution (1 mL) was added dropwise to a vial containing 7 ml of distilled water under vigorous stirring. After nanoparticle formation, the solution was dialyzed against water to remove organic solvents.

### Static Light Scattering.

Nanoparticle molar mass and aggregation number were measured via static light scattering using a Dawn Heleos II (Wyatt Technology) with detector voltages normalized with dextran sulfate (15-20 kDa). Eight sample concentrations (0.08, 0.07, 0.06, 0.05, 0.04, 0.03, 0.02, 0.01 mg/mL) were injected via a syringe pump (KD Scientific, Gemini 88) at 0.4 mL/min. The weight-averaged molar mass was obtained via Zimm plot analysis in Astra with dn/dc values estimated based on the PEG mass fraction. Aggregation numbers were calculated by dividing the nanoparticle molar mass by the bottlebrush molecular weight.

¹H NMR T₂ Relaxation.

Nanoparticles (4 mg) were washed with D₂O three times through a Amicon filter tube (100 kDa MCWO), reimmersed in 700 μL D₂O, and transferred to a 5 mm NMR tube. NMR T₂ relaxation time measurements were performed on at 500 MHz Agilent DD2 NMR spectrometer using the Carr-Purcell-Meiboom-Gill pulse sequence with a recycle delay of at least five times T₁ at 25° C. The peak intensity at 3.6 ppm (PEG backbone protons) and 3.3 ppm (methoxy end group protons) were fitted via nonlinear least-square fitting to a biexponential function to obtain relaxation times and fraction of fast and slow relaxing protons in Matlab (Mathworks).

### PEG Exposure Measurement.

A 1H NMR spectrum with 10 s relaxation delay of purified nanoparticles in D₂O containing 0.1% w/w trimethylsilylpropanoic acid (TSP) was obtained. The samples were transferred to a separate vial and 300 μL nanoparticle solution was mixed with 1.2 mL acetone-do followed by sonication for 1 min. The resulting mixture was transferred back to a 5 mm NMR tube and the 1H NMR spectrum was recorded. The amount of PEG exposure was determined by calculating the ratio of the PEG peak integral at 3.6 ppm normalized to the TSP peak at 0 ppm before and after addition of acetone-d₆.

### Cryo-TEM.

Nanoparticles were concentrated to approximately 20 mg/mL using Amicon filter tubes (100 kDa MWCO). After glow-discharging Quantifoil holey carbon 300 mesh copper grids (Electron Microscopy Sciences) using a PELCO easiGlow, a FEI Vitrobot cryo plunger (ThermoFisher Scientific) was used to vitrify nanoparticles in liquid ethane. Sample grids were kept in liquid nitrogen at all times and mounted onto a cryogenic holder (Gatan 626) before imaging. Cryo-TEM images were obtained on a FEI Talos L120C (ThermoFisher Scientific) operated at an acceleration voltage of 120 kV and under low dose conditions at about 4 μm defocus. Nanoparticle core diameters were determined via the ParticleSizer ImageJ plugin. The outer brush radius was obtained by measuring the width of at least 50 individual bottlebrush polymers per sample in ImageJ.

### BSA Adsorption.

Concentrated nanoparticles in water were diluted with 10×PBS to a final concentration of 2 mg/mL and 1×PBS. BSA-FITC was dissolved in PBS (2 mg/mL) and 200 μL mixed with 1 mL of nanoparticle solution in an Eppendorf tube and incubated at 37° C. for 1 h. The samples were washed with PBS four times by centrifuging at 21,130 g at 4° C. for 1 h in a microcentrifuge and carefully removing the supernatant before the pellet was redispersed in 1 mL cold PBS. After the last washing step, 1.5 mL DMF was added and pellets redispersed by 5 min sonication in a sonication bath. The fluorescence emission intensity was measured at 530 nm and an excitation wavelength of 480 nm using a fluorimeter (Perkin Elmer, LS55). Calibration standards were prepared by serial dilution of BSA-FITC in DMF. The experiment was performed in quadruplicates.

### Cell Uptake.

RAW 264.7 and HeLa were cultured in T75 cell culture flasks with high-glucose Dulbecco's modified Eagle's medium (DMEM) containing 10% fetal bovine serum (FBS), and 1% penicillin-streptomycin (10,000 U/ml of penicillin and 10,000 g/ml of streptomycin, Gibco-BRL) at 37° C. in 5% CO2 humidified atmosphere for 24 h. The cells were detached from the culture flask using a scrapper for RAW 264.7 or trypsin-EDTA solution for Hela cells during their exponential growth phase. Cell suspensions (0.5 mL per well) at a concentration of 10⁵ cells/ml in growth media were transferred to 24-well plate and incubated overnight. The cellular media was removed and 0.45 ml of fresh DMEM was added before nanoparticle treatment. Nanoparticle solutions were prepared in PBS with their concentration normalized based on their fluorescence intensity relative to Sy-M60 (200 μg/ml in growth media) and 50 μl sample was added to each well. The cells were further incubated at 37° C. for 24 h and rinsed with PBS to remove residual nanoparticles. Following treatment with trypsin-EDTA, cells were collected for flow cytometry analysis using a flow cytometer (Attune NxT, Invitrogen).

### Results and Discussion

### Polymer Design.

BBCP synthesis was based on sequential graft-through ring-opening metathesis polymerization (ROMP) of PEG macromonomers (PEG-MM) and PLA macromonomer (PLA-MM) containing polymerizable norbornene anchor groups due to its excellent control of BBCP composition, nearly quantitative monomer conversion, and narrow molecular weight distribution (FIG. 2A), as described by Verduzco, et al. Chem. Soc. Rev. 2015, 44, 2405-2420 and Choinopoulos, et al. Polymers. 2019, 11, 1-31. Given the controlled process of ROMP, the BBCP length and the backbone degree of polymerization (DP) of each block was precisely tuned by adjusting the molar ratio of macromonomer to initiator, i.e., the third-generation bispyridyl Grubbs catalyst (G3). PLA-MM with a number-average molecular weight (Mₙ) of 2.6 kDa was synthesized via 1,8-diazabicyclo [5.4.0]-undec-7-ene (DBU)-catalyzed ring-opening polymerization of D,L-lactide initiated off a hydroxy-norbornene derivative. Three different lengths of PEG-MM (S=1.1 kDa, M=2.3 kDa, L=3.5 kDa) were prepared by attaching methoxy-PEG-amine to a norbornene-N-hydroxysuccinimidyl-ester following Liu, et al. J. Am. Chem. Soc. 2012, 134, 16337-16344. All BBCPs were designed with a target backbone DP of 80 to obtain nanoparticles of approximately similar size.

Samples were named according to the BBCP type (Symmetric, Asymmetric, Triblock), their PEG side chain length (Short, Medium, Long) followed by the targeted PEG block backbone DP. Four symmetric BBCPs (Sy-M20, Sy-M40, Sy-M60, Sy-M70), two asymmetric (Asy-S60, Asy-L60) as well as three triblock BBCPs with terminal and interfacial PEG block side chain of different length (Tri-S40-M20, Tri-S40-L20, Tri-L40-M20) were further examined. To evaluate the effect of BBCP architecture on nanoparticle surface topography, the BBCPs were categorized based on four structural parameters: 1) ratio of backbone DP of the PEG and PLA blocks (backbone ratio), 2) side chain asymmetry of diblock BBCPs (asymmetry), 3) interfacial side chain asymmetry of triblock BBCPs with similar terminal block side chain (interfacial asymmetry), and 4) brush width of the terminal PEG block (brush width).

### Polymer Characterization.

BBCPs were characterized via size exclusion chromatography (SEC), preferably GPC, and NMR. SEC analysis revealed successful chain extension with monomodal molecular weight distribution and a negligible amount of unreacted intermediate bottlebrush polymer after the addition of the second and third block, respectively (FIG. 2B). Near-complete consumption of macromonomer after quenching the polymerization with ethyl vinyl ether was confirmed by the absence of the norbornene vinyl peak at 6.3 ppm in 1H NMR. The BBCP molecular weight and backbone DP was determined via a combination of SEC equipped with a multi-angle light scattering detector (SEC-MALS) in DMF (1 g/mL LiBr) of the PEG block before the addition of PLA-MM (dn/dc=0.055) and 1H NMR of the final BBCP (Table 1). All BBCPs were obtained at close to the targeted DP for the individual block backbone length, as well as the total backbone length, with low dispersity.

### Nanoparticle Formation.

BBCP self-assembly was first attempted via a direct dissolution method proposed by Fenyves et al. J. Am. Chem. Soc. 2014, 136, 7762-7770, which involves the hydration of a polymer film above the glass transition temperature (Tg) of PLA (48° C.) to facilitate micelle formation. However, BBCPs with large PLA content did not form nanoparticles based on dynamic light scattering (DLS) and visible aggregates. It was hypothesized that BBCPs with unfavorable symmetry do not readily self-assemble into spherical nanoparticles due to the chain stretching required for the hydrophobic block to adopt a cone-shaped morphology. Thus, an ultrasonication step was added to kinetically drive the self-assembly of BBCPs into their thermodynamically stable morphology through acoustic cavitation-induced reorganization of BBCPs in a metastable state. In particular, short sonication (15 min) resulted in the lowest polydispersity (PdI) and size without affecting the BBCP whereas signs of polymer degradation were observed in SEC and NMR at longer sonication times.

Using this modified method, highly monodisperse nanoparticles around 100 nm in size according to DLS (PdI<0.2) were obtained except for Sy-M20 (Table 2). Despite extensive sonication of Sy-M20, large aggregates remained in solution and no nanoparticle formation was observed in DLS. It is hypothesized that the small amount of PEG in combination with the long backbone of the PLA block could not accommodate a spherical morphology and alternative structures of larger size formed (Yang et al Macromolecules 2019, 52, 7042-7051). Notably, a diameter of 100 nm corresponded to approximately twice the length of a BBCP with 80 macromonomers polymerized (˜0.6 nm per repeat unit of the backbone), indicating that nanoparticles with uniform micellular structure were formed and that their size can be well controlled by the BBCP backbone DP (Walsh et al Proc. Natl. Acad. Sci. U.S.A 2019, 116, 1538-1542). After nanoparticle formation and DLS analysis, the samples were concentrated and washed with water three times using a centrifuge filter which effectively removed any trace amount of macromonomer and intermediate PEG bottlebrush polymer that did not get incorporated into the nanoparticle, as indicated by the absence of low molecular weight peaks in the SEC traces of purified nanoparticles.

### Nanoparticle Characterization.

A slightly negative zeta potential was observed for all nanoparticles with increasingly more negative values for nanoparticles with lower PEG content (Table 2). Static light scattering was used to examine the effect of BBCP architecture on nanoparticle molar mass and aggregation number. Despite only small variations in side chain length and block backbone DP ratio, a wide range of aggregation numbers was observed (Table 2). The aggregation number for the series of symmetric BBCPs with varying backbone ratio (Sy-M40, Sy-M60, Sy-M70) increased proportionally as predicted from the increase in PLA backbone DP according to the cone-filling model. For diblock BBCPs with constant PLA backbone length (Asy-S60, Sy-M60, Asy-L60), increasing the length of the interfacial block PEG side chain led to a drastic decrease in aggregation number from 1958 to 59 following the increase in side chain asymmetry.

A similar trend was observed for the series of BBCPs with short 1.1 kDa PEG as the terminal block side chain (Asy-S60, Tri-S40-M20, Tri-S40-L20) where the aggregation number decreased as a longer interfacial block side chain was used. Triblock BBCPs had larger aggregation numbers than corresponding diblock BBCPs with a side chain length similar to the triblock interfacial block side chain, implying that the hydrophilic terminal block side chain on the nanoparticle surface has an effect on self-assembly not considered in the cone-filling model but predicted from dissipative particle dynamics simulations (Yang 2019; Wessels, et al. Soft Matter 2019, 15, 3987-3998; Lyubimov, et al Macromolecules 2018, 51, 7586-7599.).

The results show that controlled interfacial side chain asymmetry can be successfully applied to increase the aggregation number for nanoparticles made from BBCPs of large terminal PEG block brush width by incorporating a shorter PEG side chain block at the PLA interface, as in Asy-L60 and Tri-L40-M20, respectively.

Cryogenic transmission electron microscopy (Cryo-TEM) was used to image nanoparticles in their native hydrated state showing highly monodisperse nanoparticles with individual PEG bottlebrush polymers protruding from the solid PLA core (FIG. 3A, 3B). Differences in brush width between 1.1 kDa, 2.3 kDa, 3.5 kDa PEG side chains as well as nanoparticle surface topography were visible. The cryo-TEM images closely match DLS and SLS analysis in terms of nanoparticle size and aggregation number with the exception of Asy-S60. A mixture of spherical and elongated nanoparticles was observed, whereas a unimodal peak in DLS indicates a monodisperse size distribution with a hydrodynamic diameter close to that of the spherical nanoparticles. Asy-S60 exhibits uniform spherical morphology after self-assembly but coalesces into elongated nanoparticles during cryo-TEM sample preparation.

Nanoparticle core diameters obtained via cryo-TEM ranged between 15 and 50 nm. For symmetric BBCPs Sy-M40, Sy-M60, and Sy-M70, the core size is directly proportional to the PLA block backbone DP and approximately doubles as the PLA block backbone length is doubled. However, the other nanoparticles with comparable PLA block backbone DP of approximately 20 varied in their core diameter beyond what can be expected solely from the differences in backbone DP according to SEC analysis. Core size and hydrodynamic diameter seem to be correlated to the aggregation number where a larger aggregation number leads to an increase in size, presumably due to the increased stretching of the bottlebrush backbone and PLA tail.

The micellular core-shell structure of the nanoparticles observed in cryo-TEM was confirmed by determining the fraction of PEG that is located in the solvated shell as compared to PEG that is trapped inside the solid core via proton nuclear magnetic resonance (1H NMR) spectroscopy. 1H NMR spectra of nanoparticles in D₂O containing trimethylsilylpropanoic acid as an internal standard before and after the addition of four parts acetone-do were obtained and the solubilized PEG signals were compared to quantify the relative PEG exposure. As shown in Table 2, no considerable differences among nanoparticles were observed with nearly all PEG located in the nanoparticles shell, indicating a highly homogeneous PLA core and PEG shell.

### NMR Relaxometry.

NMR relaxometry is a powerful and readily available method that provides information about polymer chain flexibility and local chain dynamics in polymer aggregate (Zhang et al. Anal. Chem. 2017, 89, 12399-12407.). De Graaf et al. observed that linear PLA-PEG micelles in D₂O exhibit a biphasic 72 relaxation for PEG protons which was attributed to rigid, fast-relaxing PEG segments close to the hydrophobic core and flexible, slow-relaxing segments in the outer shell (De Graaf, et al. Langmuir 2011, 27, 9843-9848). It was hypothesized that similar T2 relaxation measurements could be used to distinguish between the densely crowded inner shell and the more flexible outer shell of nanoparticles assembled from BBCPs based on their different relaxation behaviors.

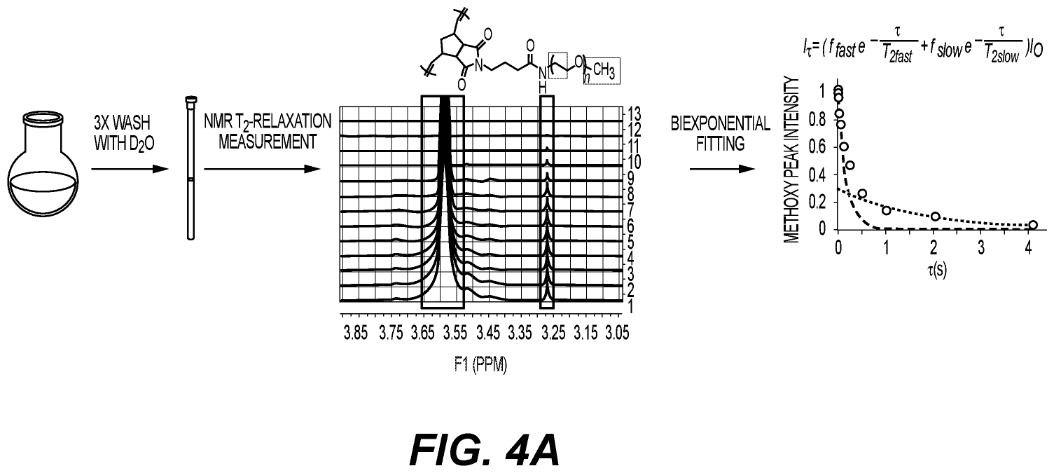

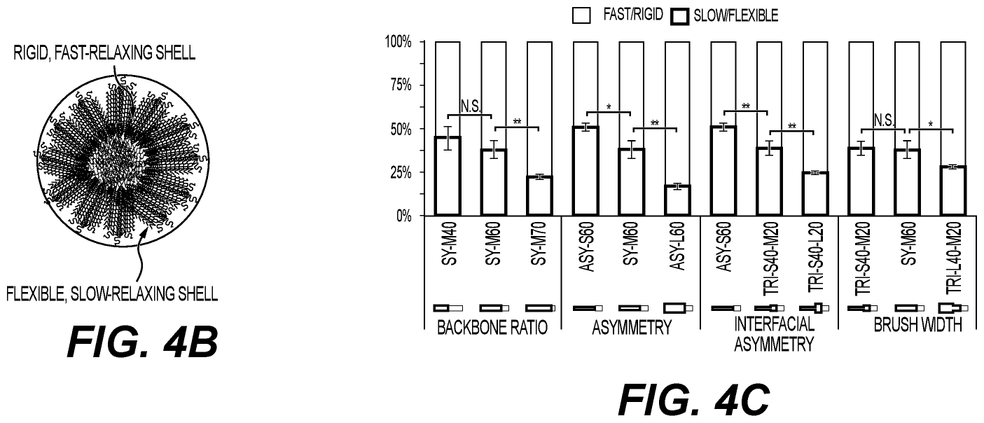

First, nanoparticles were washed with D₂O several times to completely remove non-deuterated H₂O and NMR T₂ relaxation measurements were obtained. FIGS. 4A-4C are based on NMR 72 relaxation of biphasic nanoparticle shell. (4A) Schematic illustration of NMR T₂ relaxation measurement procedure for biexponential fitting of methoxy (3.3 ppm) and PEG (3.6 ppm) proton signals. (4B) Schematic representation of nanoparticle assembled from PLA-PEG BBCPs consisting of fast-relaxing inner shell and slow-relaxing outer shell. (4C) Summary of fast/slow-relaxing PEG-methoxy end groups obtained from biexponential fitting. Data are presented as mean±SD (two-tailed t test, n=3 independent replicates). *P<0.05, ** P<0.01.

As shown in FIG. 4A, only protons in the solvated shell were visible, corresponding to PEG (3.6 ppm) and PEG methoxy end group (3.3 ppm) protons. Fitting the relaxation data to a standard monoexponential fit did not yield acceptable R² values. However, biexponential fitting assuming a fast relaxing (T_{2fast}) and a slow-relaxing (T_{2slow}) component resulted in much better fitting statistics based on Eq 1, where I represents the peak intensity, τ the relaxation delay, and f_{fast} and f_{slow} the mole fraction of fast and slow-relaxing protons, respectively.

$${I}_{τ} = ( {f}_{fast} {e}^{- \frac{τ}{{T}_{2 fast}}} + {f}_{slow} {e}^{- \frac{τ}{{T}_{2 slow}}} ) {I}_{0} \qquad ( 1 )$$

The fast-relaxing rigid component was attributed to the dense inner shell with substantial side chain overlap between adjacent bottlebrush polymers and the slow-relaxing component to the flexible highly hydrated outer shell (FIG. 4B). A PEG bottlebrush homopolymer (PEG_{2.3k}60) that was also investigated exhibited mono-exponential relaxation behavior for both PEG and methoxy protons, strongly supporting the idea that the biphasic relaxation is caused by the restricted flexibility of PEG side chains in the nanoparticle shell rather than the structure of the bottlebrush polymer.

While fitting parameters for PEG protons and methoxy protons showed similar trends, the relaxation of methoxy end group protons was used to obtain information about the extent and thickness of the inner and outer shell (FIG. 4C). In particular, the number of end group protons is directly proportional to the bottlebrush backbone DP of the hydrophilic block whereas the number of PEG protons varies with side chain length and overemphasizes the contribution of longer side chains in triblock BBCPs as well as their location within the nanoparticle shell.

The fraction of fast-relaxing end group protons decreased for symmetric BBCPs (Sy-M40, Sy-M60, Sy-M70) as the ratio of PEG to PLA backbone DP increases following the trend in aggregation number. Asy-S60, the nanoparticle with the largest aggregation number, exhibited the largest fraction of fast-relaxing end groups with 51%, indicating a highly crowded shell with considerable side chain overlap. However, increasing the side chain asymmetry of diblock BBCPs (Sy-M60 and Asy-L60) significantly reduced the fraction of fast-relaxing protons. A similar effect was observed for triblock BBCPs with larger interfacial asymmetry (Tri-S40-M20 and Tri-S40-L20), indicating that side chain asymmetry can be used to effectively control the crowding and density of the nanoparticle shell. Consequently, BBCPs with different terminal block brush widths but similar interfacial asymmetry (Tri-S40-M20, Sy-M60, Tri-L40-M20) only experienced small differences in their fraction of fast-relaxing end group protons.

The ratio of inner to outer shell thickness decreased with increasing ratio of hydrophilic to hydrophobic backbone length, consistent with the results obtained for the series of symmetric BBCPs. The relative extent of inner and outer shell for BBCPs with comparable architecture is within the range of values for the fraction of fast/slow-relaxing end group protons reported herein. The findings show that NMR relaxometry can provide a readily accessible and straightforward alternative to SANS in the study of nanoparticles formed from BBCPs.

### Surface Topography.

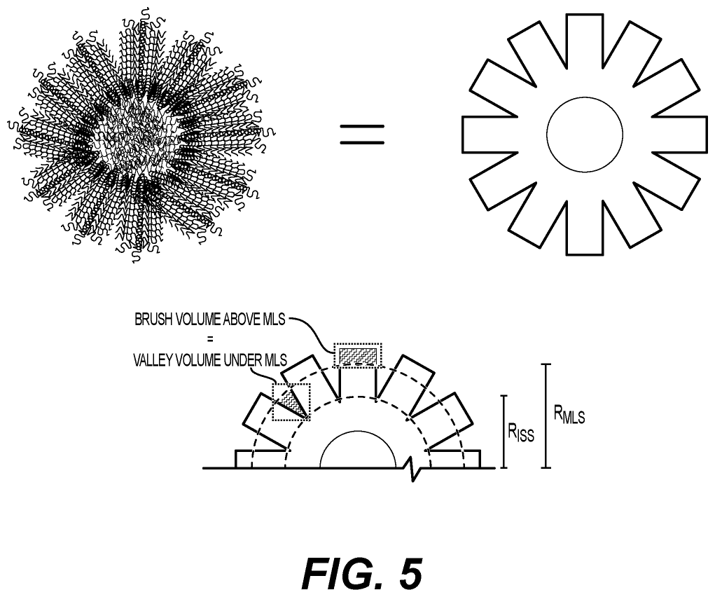

Quantitative characterization of nanoparticle surface topography (i.e., surface roughness and surface area) is a challenge due to the small characteristic length scale of the surface. Standard surface characterization methods such as atomic force microscopy and nitrogen adsorption isotherms that have been used for nanoparticles in the past are inadequate to analyze soft polymeric nanoparticles in their native hydrated state as their shell is prone to deformation and collapse upon dehydration. This study used a simple model to approximate the surface topography by depicting the nanoparticle shell as a number of individual cylinders with an overlapping portion close to the solid core (FIG. 5).

FIG. 5 is a schematic illustration of nanoparticle with hydrophobic core and hydrophilic shell approximated as overlapping cylinders. The mean-line surface radius (R_{MLS}) represents the radius where the brush volume above equals the valley volume below the MLS. The inner shell sphere radius (R_{ISS}) corresponds to the radius of the overlapping portion of the bottlebrush cylinder shell.

The radius of cylinders representing the outer segment of the PEG bottlebrush polymer (R_{brush}) was determined from cryo-TEM images and follows the trend in terminal block PEG side chain degree of polymerization (N) (Table 3). In particular, the bottlebrush radius scales with approximately N^{0.7} in agreement to the predicted scaling of N^{3/4} for bottlebrush polymers in good solvent (Verduzco Chem. Soc. Rev. 2015, 44, 2405-2420). The radius of the inner shell sphere (R_{ISS}) containing the overlapping cylinder portion was estimated based on the fraction of fast-relaxing PEG end group protons from NMR 72 relaxation. The mean line sphere radius (R_{MLS}) representing a sphere of equal volume was calculated. The arithmetic mean roughness was determined based on the sum of height deviations from the mean line sphere (MLS) divided by the MLS surface area (A) and the MLS radius (r) to account for the nanoparticle curvature according to previously reported Eq 2, where |Z(x, y)| represents the integral of the cylinder volume above the MLS and valley volume below (Chang et al RSC Adv. 2017, 7, 40255-40261).

$$roughness = \frac{1}{Ar} \int {\int }_{0}^{A} \vert Z ( x , y ) \vert dxdy \qquad ( 2 )$$

Roughness calculations show that Sy-M40 is the nanoparticle with the smoothest surface and the mean roughness substantially increases as the PEG/PLA block backbone ratio becomes larger for Sy-M60 and Sy-M70 (Table 3). Furthermore, larger interfacial PEG/PLA side chain asymmetry results in increased roughness for diblock BBCPss as well as triblock BBCPs with similar terminal block PEG side chain. Specifically, the roughness of nanoparticles based on BBCPs with short 1.1 kDa PEG side chain as the terminal block increases from 0.40 for Asy-S60 to 0.67 for Tri-S40-M20 and 0.89 for Tri-S40-L20 being the nanoparticle with the roughest surfaces. A larger terminal block brush width in Tri-L40-M20 and Sy-M60 resulted in a smoother surface compared to Tri-S40-M20. Notably, the surface roughness of nanoparticles studied herein is approximately one order of magnitude higher than values obtained via a similar method for rough silver-coated silica nanoparticles of equivalent size Chang, et al. RSC Adv. 2017, 7, 40255-40261.

The nanoparticle surface area was calculated from the surface area of the non-overlapping portion of bottlebrush cylinders as follows:

surface area per nanoparticle=N_{agg}(2πR_{brush}(Rₕ−R_{ISS})+πR_{brush}²) (3)

The surface area varied considerably between nanoparticles despite only small differences in diameter. The aggregation number strongly affected the calculated surface area, as expected from the increasing number of individual bottlebrush polymers exposed on the nanoparticle surface. For example, Asy-S60 has an approximately 14 times larger surface area than Asy-L60. Additionally, when the nanoparticle surface area is compared to that of an equivalent smooth nanoparticle with homogeneous shell density based on the MLS radius, an increase in surface area by a factor of 4.1 to 11.3, depending on the BBCP architecture, was observed. Bottlebrush polymers in the nanoparticle shell are expected to behave as impenetrable hard cylindrical surfaces, but these results demonstrate the ability of the approach to generate nanoparticles with controlled surface topography as judged by their increase in surface area and roughness.

### Example 2: BBCP Nanoparticle Protein Absorption

### Materials and Methods

An important predictor of nanoparticle in vivo performance and pharmacokinetics is the adsorption of serum proteins and formation of a protein corona on the nanoparticle surface. In particular, the total amount of protein adsorption has been found to directly correlate to blood circulation times with reduced adsorption resulting in prolonged circulation. A simple protein adsorption assay, based on FITC-labeled bovine serum albumin (BSA), was developed which is similar to the most abundant human serum protein HSA. Nanoparticles in PBS were incubated with BSA-FITC at 37° C. and washed with fresh PBS four times to remove any protein not adsorbed onto the nanoparticle surface. The fluorescence signal of protein remaining in the pellet was then measured to calculate the amount of BSA adsorbed.

### Results

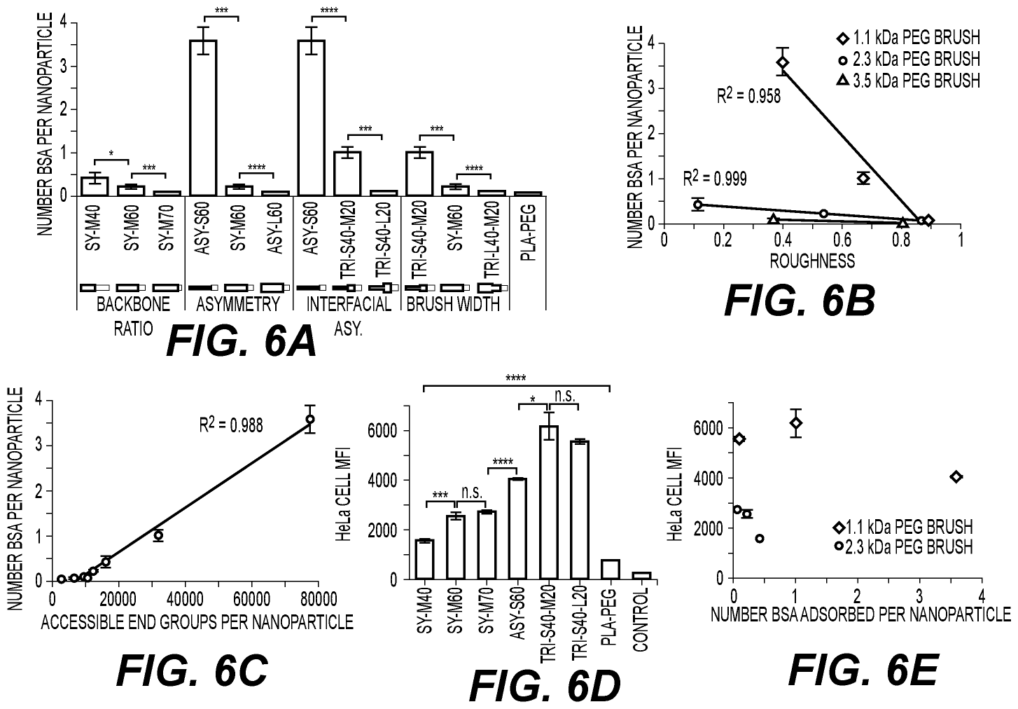

FIG. 6A-6E are graphs showing the effect of nanoparticle surface topography on BSA adsorption and cell uptake. FIG. 6A shows BSA-FITC adsorption of nanoparticles assembled from PLA-PEG BBCPs and a smooth nanoparticle control based on linear PLA-PEG. FIG. 6B shows the impact of nanoparticle roughness on the number of BSA-FITC adsorbed per nanoparticle. Trendlines are based on the molecular weight of the terminal block PEG side chain used. FIG. 6C shows the number of BSA-FITC per nanoparticle compared to the number of accessible slow-relaxing groups obtained from NMR relaxation. FIG. 6D shows the mean fluorescence intensity (MFI) of HeLa cells incubated with DiO-loaded nanoparticles. FIG. 6E shows a comparison of nanoparticle uptake into HeLa to the number of BSA adsorbed for nanoparticles with 1.1 kDa PEG side chain as the terminal block and 2.3 kDa PEG.

Considerable differences in BSA adsorption were observed between nanoparticles with the best-performing nanoparticles exhibiting virtually no adsorption, similar to a ‘stealthy’ smooth nanoparticle based on linear PLA-PEG with high PEG surface density (0.38 PEG/nm², Dₕ=87 nm). The lowest adsorption was obtained for nanoparticles with long 3.5 kDa PEG terminal block side chain (Asy-L60 and Tri-L40-M20) whereas Asy-S60 adsorbed by far the most protein per particle followed by Tri-S40-M20. Nanoparticles based on symmetric BBCPs exhibited slightly reduced BSA adsorption with larger PEG/PLA block backbone ratio. However, increasing the side chain asymmetry for diblock BBCPs as well as the interfacial asymmetry for triblock BBCPs resulted in substantial lower protein adsorption with highly significant differences between samples. Furthermore, the terminal block brush width played a significant role with the narrow Tri-S40-M20 BBCP showing increased adsorption compared to Sy-M60 and Tri-L40-M20.

The importance of terminal block brush width was also evident when BSA adsorption was compared to nanoparticle surface roughness. The number of BSA proteins adsorbed per nanoparticle linearly decreased with surface roughness with the extent dependent on the terminal block side chain length (FIG. 6B). Specifically, a shorter side chain increases the contribution of surface roughness in reducing protein adsorption. While it has been previously reported that the local curvature of rough patchy nanoparticles inhibits protein adsorption, it was speculated that the short terminal block side chain increases the flexibility of the bottlebrush backbone and may allow proteins to penetrate the nanoparticle shell more easily. However, this effect gradually diminishes as a longer terminal block side chain is incorporated and the bottlebrush backbone becomes more rigid.

In general, it is well established that protein adsorption of PEGylated nanoparticles decreases with PEG surface density due to increased steric repulsion, as PEG chains transition from mushroom to brush conformation (Cao, et al. ACS Nano 2020, 14, 3563-3575). However, as the shell of nanoparticles composed of BBCPs does not consist of a homogenous layer of flexible PEG that readily undergoes conformational changes, the protein adsorption cannot be accurately represented by simple calculation of PEG surface density. The extraordinary high density of PEG in the nanoparticle shell dictated by the bottlebrush backbone (i.e., one PEG side chain per five backbone carbon atoms) is expected to provide sufficient shielding to prevent any substantial hydrophobic interactions between proteins and the PLA core. Thus, protein adsorption is primarily driven by direct interaction between PEG and proteins. Han et al. J. Mater. Chem. B 2017, 5, 8479-8486 reported that increasing the density of PEG grafted onto a hydrophobic substrate beyond a critical grafting density results in increased protein adsorption via intermolecular protein-PEG interactions such as hydrogen-bonding. These interactions primarily occur between proteins and hydrophilic PEG methoxy head groups due to the directional dependency of hydrogen bonding (Huh et al Macromolecules 1997, 30, 1828-1835). Protein adsorption should be directly correlated to the number of PEG end groups per nanoparticle that are accessible for proteins to interact with rather than the actual surface area. The number of accessible PEG end groups in the outer nanoparticle shell was estimated based on the fraction of slow relaxing PEG end group protons (f_{slow}), the number of BBCPs per nanoparticle (N_{agg}), and number of PEG side chain per BBCP (n_{PEG,backbone}) (Eq 3).

accessible end groups per nanoparticle=(N_{agg})(n_{PEG,backbone})(f_{slow}) (3)

Comparison of BSA adsorption to the number of accessible PEG end groups resulted in a positive linear correlation (FIG. 6C), supporting the hypothesis that BSA directly interacts with PEG end groups whereas nanoparticle surface area failed to give a conclusive trend. Surprisingly, this relationship indicates that it is desirable to reduce the amount of PEG chains per nanoparticle to minimize protein adsorption for nanoparticle assembled from BBCPs. This behavior is drastically different from other PEGylated nanoparticles where protein adsorption is typically inhibited as the number of PEG chains increases.

### Example 3: Effect of Nanoparticle Surface Topography on Cell Uptake

### Materials and Methods

The effect of nanoparticle surface topography on cell uptake was investigated using a human cervical cancer HeLa cell line and a murine macrophage RAW264.7 cell line. Nanoparticles were loaded with fluorescent DiO dye to measure their uptake into cells via flow cytometry. Cells were incubated with a nanoparticle concentration normalized based on their fluorescence signal.

### Results

Similar trends in nanoparticle uptake were observed for both cell lines, with all tested nanoparticles experiencing considerably increased uptake compared to the linear PLA-PEG nanoparticle.

The effect of nanoparticle surface topography on cell uptake was investigated using a human cervical cancer HeLa cell line (FIG. 6D) Tumors harvested 24 hours, 72 hours, and 144 hours from mice after injection with Asy-XL60 and Tri-S40-XL20 were analyzed by FACS with histograms showing nanoparticle internalization in tumor-associated macrophages and tumor cells.

Nanoparticles with narrow terminal block brush width (Asy-S60, Tri-S40-M20, Tri-S40-L20) showed significantly enhanced uptake relative to nanoparticles with wider terminal block (Sy-M40, Sy-M60, Sy-M70). Notably, the uptake of Tri-S40-M20 and Tri-S40-L20 into HeLa cells approximately tripled compared to Sy-M40 and increased by a factor of almost 7 relative to the smooth PLA-PEG nanoparticle control. Within the series of nanoparticles with similar terminal block, an increase in surface roughness resulted in enhanced cell uptake. This is in agreement with reports that nanoparticle surface asperities greatly lower repulsive interactions between negatively-charged cell membranes and hydrophilic polymers such as PEG, resulting in enhanced adhesion to cells. Furthermore, the effect was more pronounced with smaller asperity size explaining the higher uptake of nanoparticles with narrow brush width.

Next, the uptake into HeLa cells was compared to the amount of BSA adsorbed. Results are shown in FIG. 6E.

Typically, low protein adsorption of PEGylated nanoparticles is correlated with reduced cell uptake as steric effects and hydrophilicity of a high-density PEG shell also inhibits interactions with cell membranes. However, for nanoparticles tested in this study, a high cell uptake was observed even for nanoparticles that experienced low BSA adsorption. Specifically, Tri-S40-L20 showed a comparable high level of HeLa cell uptake to that of Tri-S40-M20 while being among the lowest BSA adsorbing nanoparticles. In addition, Tri-S40-L20 exhibited substantially enhanced uptake compared to the conventional smooth PLA-PEG nanoparticle but similarly low BSA adsorption. These results indicates that improvement of protein adsorption and cell uptake does not necessarily present a trade-off as commonly observed for PEGylated nanoparticles. In fact, protein adsorption and cell uptake were simultaneously improved through surface roughness and terminal block brush width, demonstrating the importance of surface topography in nanoparticle research.

### Example 4: In Vivo Uptake of Block Brush Polymers into Tumors

### Materials and Methods

4T1 cells (5×10⁵ cells in PBS) were injected into 6 weeks old female BALB/c mice subcutaneously near the mammary gland. After 10 days, nanoparticles (10 mg/mL in PBS) labelled with 0.4 wt % Cy5-norbornene were injected retro-orbitally at a dose of 60 mg/kg. In vivo IVIS images were taken 1, 24, 48, 72, 120, and 144 hours after injection.

Two mice per nanoparticle were sacrificed after 24, 72, and 144 hours and their organs harvested for ex vivo IVIS. Subsequently, the tumor tissue was homogenized using a razor blade and digested (0.1% collagenase and 0.2% dispase type II in RPMI buffer). After washing the cells with EDTA buffer (2% BSA), red blood cells were removed using red blood cell lysis buffer followed by another washing step. The cells were resuspended in PBS at a concentration of 4 million cells/mL and stained with Zombie UV as well as fluorophore-conjugated antibodies. Flow cytometry analysis was performed on a CytoFlex LX (Beckman Coulter) at the Yale Flow Cytometry Facility.

To assess if nanoparticles were able to penetrate into the tumor and achieve cell internalization, the tumor tissue was digested and analyzed the nanoparticle cell uptake via flow cytometry. CD11b+F4/90+ cells corresponding to tumor-associated macrophages and EPCAM+ cells (4T1 tumor cells) were imaged.

IVIS Cy5-fluorescence images of nanoparticle standards were prepared from Asy-XL60 and Tri-S40-XL20. In vivo imaging of Cy5-fluorescence of mice injected with Cy5-labelled nanoparticles injected with Asy-XL60 and Tri-S40-XL20 was performed 1 hour, 24 hours, 48 hours, 72 hours, 120 hours and 144 hours after injection. The location and size of the subcutaneous 4T1 tumors was determined by imaging. Spleen, kidney, liver, lung, heart and tumor were then excised 72 hours and 144 hours after mice were injected with Asy-XL60 and Tri-S40-XL20 and imaged.

### Results

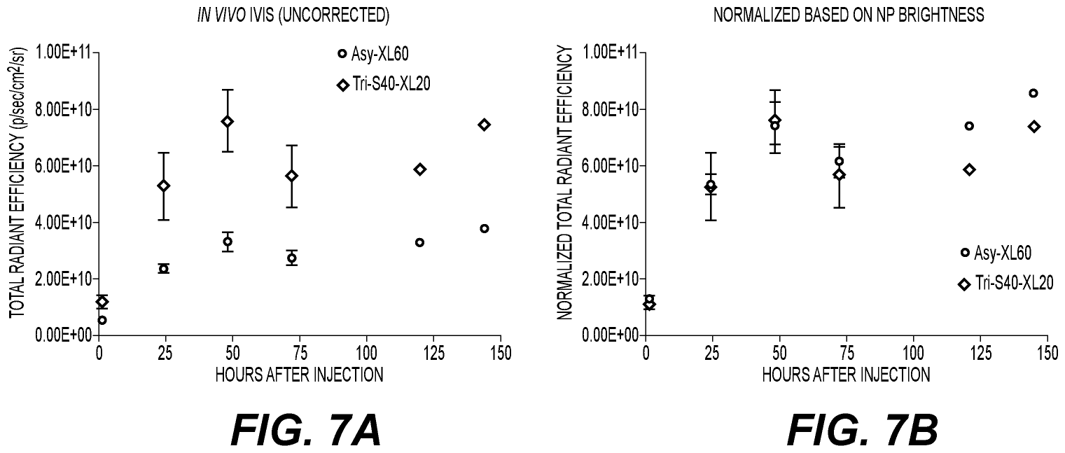

Cy5-labelled nanoparticles based on BBCPs Asy-XL60 (5 kDa PEG side chain) and Tri-S40-XL20 (1.1 kDa PEG side chain for terminal block and 5 kDA for middle block) were injected retro-orbitally into tumor-bearing mice and the nanoparticle tumor accumulation was monitored via in vivo IVIS. FIGS. 7A and 7B are graphs showing quantification of nanoparticle tumor accumulation based on IVIS images of subcutaneous 4T1 tumors after injection with Cy5-labelled nanoparticles Asy-XL60 and Tri-S40-XL20. Asy-XL60 contains 5 kDa PEG side chains and Tri-S40-XL20 is based on 1.1 kDa PEG side chains for the terminal block and 5 kDa PEG for the interfacial middle block. FIG. 7A shows total IVIS radiant efficiency of tumor area not corrected for differences in nanoparticle brightness. FIG. 7B shows IVIS radiant efficiency normalized based on the brightness of Tri-S40-XL20.

Both nanoparticles showed gradually increasing accumulation in tumors throughout the first 48 hours after injection with a small reduction in tumor signal after 72 hours. Unexpectedly, the tumor accumulation increased again for later time points and reached its maximum for Asy-XL60 at the end point of the study after 144 hours indicating remarkably enhanced nanoparticle accumulation and retention in tumors. Comparison between the nanoparticles by normalizing the IVIS data based on nanoparticle fluorescence (FIG. 7B) shows negligible differences in tumor accumulation for earlier time points (i.e., 1, 24, and 48 hours) but higher signal for Asy-XL60 after 120 and 144 hours possibly due to differences in nanoparticle tumor retention and tumor cell uptake which occurs over longer timescales.

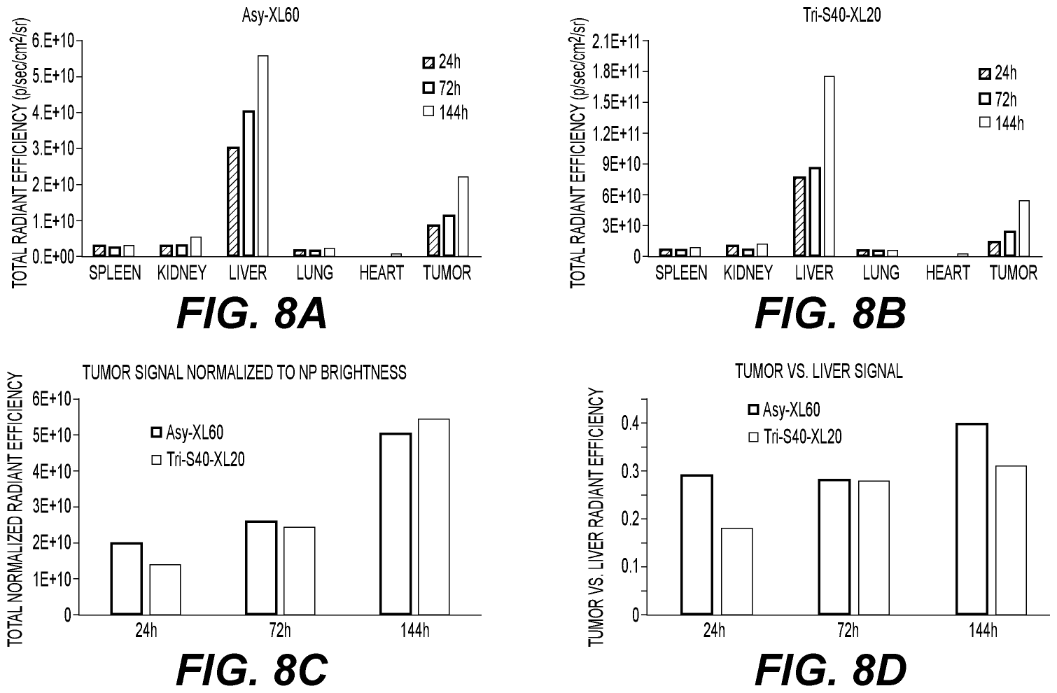

Next, the nanoparticle biodistribution and selectivity of tumor accumulation via ex vivo IVIS of organs harvested from mice 24, 72, and 144 hours after injection were determined. Both nanoparticles exhibited accumulation primarily in liver and tumor which substantially increased over time (FIGS. 8A and 8B). While only small differences in tumor accumulation were observed between nanoparticles (FIG. 8C), Asy-XL60 experienced a higher selectivity for tumor accumulation compared to liver (i.e., main nanoparticle clearance organ) than Tri-S40-XL20 (FIG. 8D). Nevertheless, both nanoparticles showed remarkably high selectivity towards tumor accumulation.

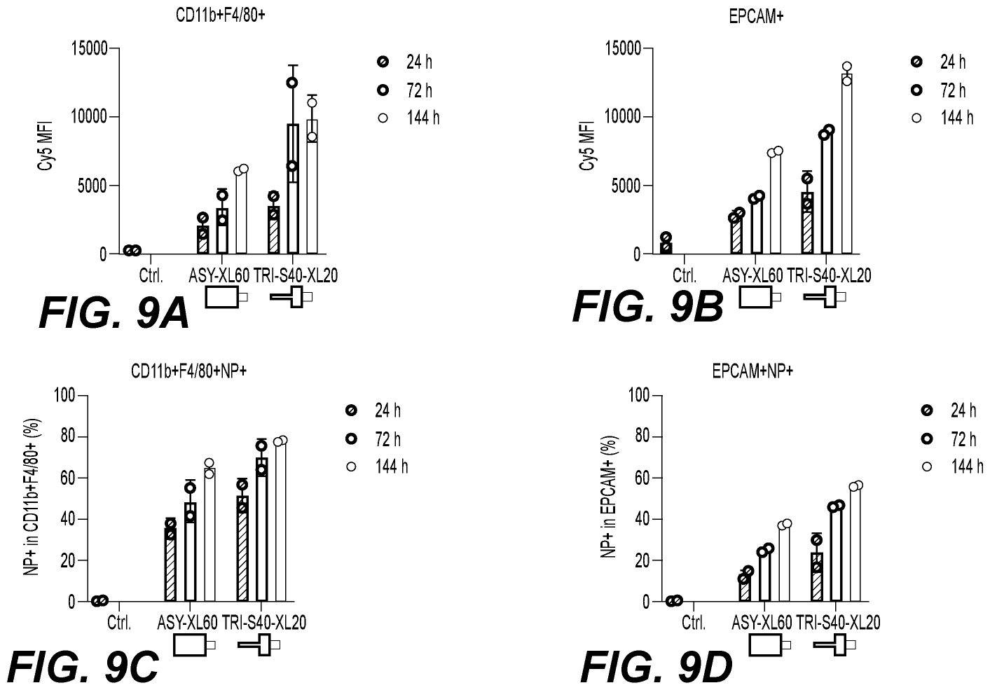

Increasingly high uptake into tumor cells (EPCAM+) and tumor-associated macrophages (CD11b+F4/80+) was observed over time (FIGS. 9A-9D). Tri-S40-XL20 outperformed Asy-XL60 in terms of tumor cell uptake with approximately 60% fluorescent-positive cells (compared to ˜40% for Asy-XL60) in agreement with the observed enhanced in vitro cell uptake of nanoparticles formed from triblock BBCPs with short PEG on the nanoparticle surface. Furthermore, both nanoparticles exhibited high internalization into tumor-associated macrophages (˜80% fluorescent-positive cells) which have been reported to enable enhanced nanoparticle tumor penetration.

Nanoparticle drug delivery to solid tumors via passive targeting has been extensively studied as a strategy to improve the efficacy of therapeutics. In particular, long-circulating nanoparticles exhibit enhanced tumor accumulation by avoiding clearance via the mononuclear phagocytosis system, allowing them to accumulate to a significant degree in the tumor microenvironment due to the enhanced permeability and retention (EPR) effect.

However, while these stealthy properties are desirable to achieve maximum accumulation, the same characteristics prevent the nanoparticles from penetrating into the tumor, and can also prevent their subsequent internalization by tumor cells. Internalization of drug-loaded nanoparticles can greatly enhance the activity of many drugs, so identifying long-circulating nanoparticles that are also internalized by tumor cells is important.

Based on the high in vitro cell uptake and long circulation half-lives of nanoparticles demonstrated in the examples, the tumor accumulation and in vivo tumor cell uptake was studied using an orthotopic subcutaneous 4T1 breast cancer model in mice. The results demonstrate that controlling the surface topography of nanoparticles should provide benefits for the delivery of nucleic acid and chemotherapeutic agents currently limited by unfavorable pharmacokinetics and low cell uptake.

### Example 5: Pharmacokinetics of Nanoparticles

Improving the pharmacokinetics of nanoparticle drug carriers has been extensively studied as a means to increase the efficacy of drugs as well as achieve selective accumulation in solid tumors via passive targeting. Coating nanoparticles with PEG represents a well-established strategy to improve the pharmacokinetics of nanoparticles by reducing clearance via the mononuclear phagocytosis system. However, even in the presence of PEG, the circulation time of conventional nanoparticles is limited and typically much shorter than that of red blood cells. Increasing the PEG surface density and coating the surface with a second layer of PEG have been investigated as methods to further optimize PEG-coatings but have only resulted in marginal improvements in pharmacokinetics.

The effect of nanoparticle surface topography and BBCP architecture on nanoparticle pharmacokinetics in mice and in tumors was studied.

### Materials and Methods

All animal procedures were performed in accordance with the guidelines and policies of the Yale Animal Resource Center and approved by the Institutional Animal Care and Use Committee of Yale University. Cy3-labelled nanoparticles made as described in Example 1 were suspended in phosphate buffered saline (“PBS”) (5 mg/mL) and administered via retroorbital injection (20 mg/kg) into 6-8 weeks old female C57Bl/6J mice purchased from Jackson Laboratory anesthetized using an isoflurane chamber.

After the injection, a scalpel was used to cut a small tail nick. Blood samples (2 μL) were collected from the tail nick after 0.5, 1, 2, 4, 8, 12, 24, 48 h, immediately mixed with a heparin solution (2 μL of 1000 USP/mL heparin solution in 8 μL PBS) to prevent clotting and flash-frozen using dry ice. The samples (10 μL) and nanoparticles standards were then transferred to a 384-well plate and imaged using an EVOS FL Auto 2 Cell Imaging System, with standard RFP filters and Olympus superapochromat 20×/0.75 NA objective.

The nanoparticles were labelled with Cy3 fluorescent dye by incorporating 0.2 wt % Cy3-norbornene monomer during ROMP. The nanoparticles were then injected at a dose of 20 mg/kg and the pharmacokinetics were analyzed. The fluorescence intensity of each well was quantified using Matlab and pharmacokinetic parameters calculated using one-compartmental fitting.

The circulation half-lives over 48 hours in blood (μg/ml) was measured for Tri-S40-M20; Tri-S40-L20; Tri-L40-M20, and control linear PLA-PEG.

The circulation half-life, area under the curve and in vitro HeLa cell MFI from 0 to 40 hours was assessed to compare in vitro HeLa cell uptake relative to circulation half-life showing concentration in blood (μg/ml) relative to control 1.1 kDa PEG brush; 2.3 kDa PEG brush and linear PLA-PEG.

### Results

FIGS. 10A-10D are graphs of the pharmacokinetic profiles of Cy3-labelled nanoparticles formed from BBCPs (FIG. 10A, Sy-M40; FIG. 10B, Sy-M60, FIG. 10C, Sy-M70, and FIG. 10D, Asy-L60).

FIGS. 11A-11D are graphs of the circulation half-lives (over 48 hours) showing concentration in blood (μg/ml) (FIG. 11A, Tri-S40-M20; FIG. 11B, Tri-S40-L20; FIG. 11C, Tri-L40-M20, and control linear PLA-PEG. In particular, Asy-L60 exhibited an approximately three times longer circulation half-life than the linear PLA-PEG nanoparticle with approximately 40% of the initial dose still circulating after 48 hours (FIGS. 11A-11D). The circulation half-life appeared to be dependent on the PEG side chain length with a longer side chain leading to extended circulation. Unexpectedly, nanoparticles formed from triblock BBCPs, Tri-S40-M20 (FIG. 11C) and Tri-S40-L20 (FIG. 11B), were not rapidly cleared from circulation despite the short PEG side chain length used for the terminal BBCP block. Increasing the side chain length of the interfacial PEG block in Tri-S40-L20 led to a longer circulation half-life, substantially outperforming the linear PLA-PEG nanoparticle control (FIG. 11D). Furthermore, there was considerable improvement in the area under the curve (AUC_{0-48 h}) for all samples compared to linear PLA-PEG, with the best-performing nanoparticles exhibiting a four-fold increase in AUC_{0-48 h}.

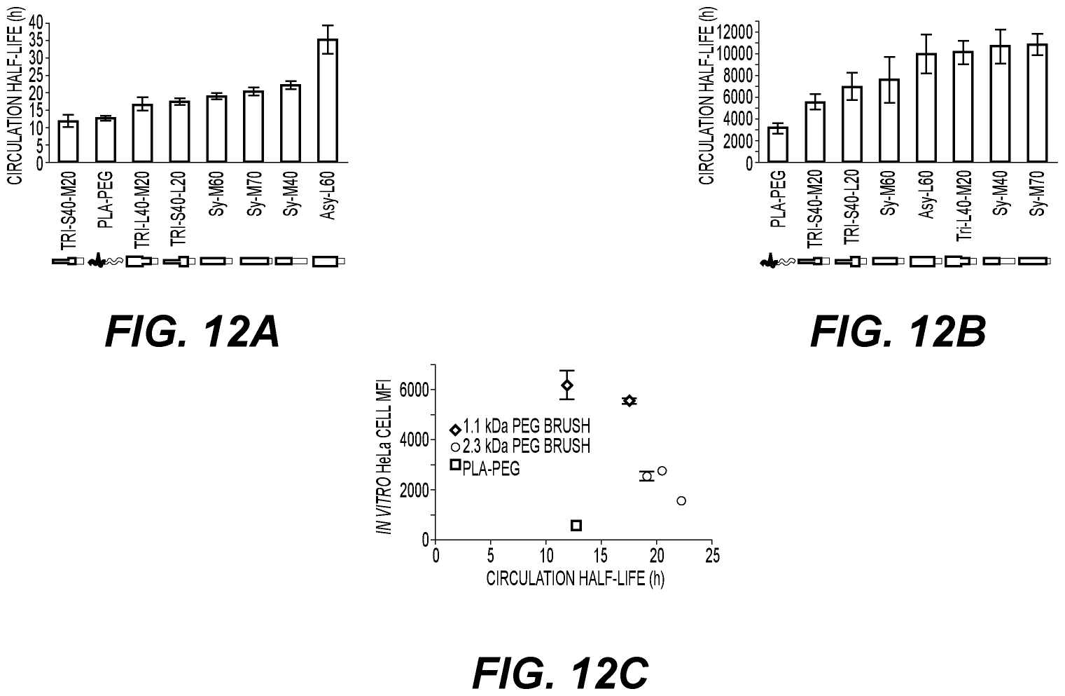

FIGS. 12A-12C are graphs of the circulation half-life (FIG. 12A), area under the curve (FIG. 12B) and in vitro HeLa cell MFI (FIG. 12C) from 0 to 40 hours comparing in vitro HeLa cell uptake relative to circulation half-life showing concentration in blood (μg/ml) relative to control 1.1 kDa PEG brush; 2.3 kDa PEG brush and linear PLA-PEG.

Remarkably, nanoparticles that exhibited high in vitro HeLa cell uptake still maintained long circulation half-lives (FIGS. 12A-12C). The nanoparticle with the roughest surface, Tri-S40-L20, showed a 7-fold increase in HeLa cell uptake relative to the smooth linear PLA-PEG nanoparticle control while being among the longest circulating nanoparticles.

These results show that high cell uptake does not necessarily lead to enhanced nanoparticle clearance via increased macrophage uptake as commonly observed.

### SUMMARY

In summary, a method to control the surface topography of PEGylated nanoparticles based on PLA-PEG BBCP building blocks has been developed which allows tuning of BBCP architecture to produce monodisperse nanoparticles with highly predictable surface topography controlled by a number of structural parameters. In vitro experiments demonstrate that nanoparticle surface topography represents an important factor in controlling protein adsorption and cell uptake that has previously not been adequately addressed. Nanoparticles with rough surface and narrow terminal brush width exhibited low protein adsorption while still maintaining high cell uptake compared to conventional smooth nanoparticles assembled from linear PLA-PEG block copolymers. As long-circulating PEGylated nanoparticles with high-density PEG shell typically suffer from low cell uptake and limited drug delivery efficiency, optimization of nanoparticle surface topography provides a strategy to improve the performance of PEGylated nanoparticles in drug delivery. The approach enables the facile generation of nanoparticles with hierarchically functionalized surfaces that can be potentially utilized for other biomedical applications.

1. Nanoparticles comprising a surface topography comprising shape-persistent amphiphilic bottlebrush block copolymer (BBCP), with controlled brush width, hydrophilic/hydrophobic block backbone length, and/or interfacial asymmetric bottle brush polyalkylene oxide polymers.

2. The nanoparticles of claim 1 comprising terminal polyalkylene oxide (PAO) block co-polymer brushes, wherein the terminal copolymer brushes are copolymers of PAO with polymers selected from the group of polyhydroxy acids consisting of polylactic acid, polyglycolic acid, polylactide-co-glycolide, and copolymers thereof.

3. The nanoparticles of claim 1 wherein the block copolymers are synthesized by ring-opening methathesis polymerization (ROMP) using PEG-norbornene and PLA-norbornene initiated by Grubbs catalyst.

4. The nanoparticles comprising a triblock BBCP with interfacial PEG block and a terminal PEG block of different side chain length and/or molecular weight to control brush width and symmetry.

5. The nanoparticles of claim 4 wherein the BBCP comprises 1, 2.3, and/or 3.5 kDa PEG side chains and 1 to 10 kDa polyhydroxy acid side chains.

6. The nanoparticles of claim 1 wherein the BBCP comprises asymmetric terminal polymers and side chains, wherein the asymmetry is determined by the ratio of interfacial PAO side chain length/MW and PLA side chain length/MW.

7. The nanoparticles of claim 6 wherein the ratio of hydrophilic/hydrophobic block backbone length (degree of polymerization) is 70/10, 60/20. 40/40 (hydrophilic/hydrophobic block backbone DP) based on a representative particle size of 100 nm.

8. The nanoparticles of claim 1 comprising therapeutic, prophylactic, or diagnostic agent.

9. The shape-persistent amphiphilic bottlebrush block copolymer (BBCP), with controlled brush width, hydrophilic/hydrophobic block backbone length, and/or interfacial asymmetry asymmetric bottle brush polyalkylene oxide polymers of claim 1.

10. A method for delivering therapeutic, prophylactic or diagnostic agent comprising administering the nanoparticles of claim 8.

11. The method of claim 10 wherein the nanoparticles are administered systemically.
